SEQUENCE LISTING (41 sequences)

SEQ ID NO: 1 - protein, 359 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 2 - protein, 350 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 3 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 4 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 5 - protein, 252 aa, Homo sapiens
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 6 - protein, 244 aa, Homo sapiens
MGPPSAAPRGGHRPWQGLLITASLLTFWHPPTTVQFTIEALPSSAAEGKDVLLLACNISETIQAYYWHKGKTAEGSPLIAGYITDIQANIPGAAYSGRETVYPNGSLLFQNITLEDAGSYTLRTINASYDSDQATGQLHVHQNNVPGLPVGAVAGIVTGVLVGVALVAALVCFLLLSRTGRASIQRDLREQPPPASTPGHGPSHRSTFSAPLPSPRTATPIYEELLYSDANIYCQIDHKADVVS

SEQ ID NO: 7 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 8 - protein, 344 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 9 - protein, 265 aa, Homo sapiens
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGFYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 10 - protein, 350 aa, Homo sapiens
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALII

SEQ ID NO: 11 - protein, 6 aa, Homo sapiens
SSHGMS

SEQ ID NO: 12 - protein, 6 aa, Homo sapiens
SFYGMS

SEQ ID NO: 13 - protein, 6 aa, Homo sapiens
SDYYLY

SEQ ID NO: 14 - protein, 17 aa, Homo sapiens
TISSGGTYTYYPDSVKG

SEQ ID NO: 15 - protein, 17 aa, Homo sapiens
TFSGGGNYTYYPDSVKG

SEQ ID NO: 16 - protein, 17 aa, Homo sapiens
TISVGGGNTSYPDSVKG

SEQ ID NO: 17 - protein, 12 aa, Homo sapiens
HDFDYDAAWFAY

SEQ ID NO: 18 - protein, 11 aa, Homo sapiens
HGGLPFYAMDY

SEQ ID NO: 19 - protein, 11 aa, Homo sapiens
GLTTGPAWFAY

SEQ ID NO: 20 - protein, 10 aa, Homo sapiens
SANSSVSYMY

SEQ ID NO: 21 - protein, 12 aa, Homo sapiens
SVSSSISSSNLH

SEQ ID NO: 22 - protein, 17 aa, Homo sapiens
KSSQSLLNSSNQKNYLA

SEQ ID NO: 23 - protein, 11 aa, Homo sapiens
RASQKISGYLS

SEQ ID NO: 24 - protein, 7 aa, Homo sapiens
LTSNLAS

SEQ ID NO: 25 - protein, 12 aa, Homo sapiens
SVSSSISSSNLH

SEQ ID NO: 26 - protein, 7 aa, Homo sapiens
FASTRES

SEQ ID NO: 27 - protein, 7 aa, Homo sapiens
AASTLDS

SEQ ID NO: 28 - protein, 9 aa, Homo sapiens
QQWSSNPPT

SEQ ID NO: 29 - protein, 9 aa, Homo sapiens
QQWSSHPFT

SEQ ID NO: 30 - protein, 9 aa, Homo sapiens
QQHYSTPWT

SEQ ID NO: 31 - protein, 10 aa, Homo sapiens
LQYASSLMYT

SEQ ID NO: 32 - protein, 20 aa, Homo sapiens
CSLFTCQNGIVWTNGTHVTY

SEQ ID NO: 33 - protein, 16 aa, Homo sapiens
CSLFTSGSGRVEHRGW

SEQ ID NO: 34 - protein, 20 aa, Homo sapiens
CPYSGCKEALIRTDGMRVTS

SEQ ID NO: 35 - protein, 16 aa, Homo sapiens
CPYSGSGSGRIEVPGW

SEQ ID NO: 36 - protein, 20 aa, Homo sapiens
CPVFECGNVVLRTDERDVNY

SEQ ID NO: 37 - protein, 6 aa, Artificial Sequence
  Description of Artificial Sequence Synthetic 6xHis tag
HHHHHH

SEQ ID NO: 38 - protein, 17 aa, Homo sapiens
CPVFESGSGRIQIPGIM

SEQ ID NO: 39 - protein, 18 aa, Homo sapiens
CPVFESGSGRIQIPGIMN

SEQ ID NO: 40 - protein, 19 aa, Homo sapiens
CPVFESGSGRIQIPGIMND

SEQ ID NO: 41 - protein, 4 aa, Homo sapiens
SGSG

(19) United States
(12) Patent Application Publication
(10) Pub. No.: US 2018/0298095 A1
(43) Pub. Date: Oct. 18, 2018

(54) INTERACTIONS BETWEEN CEACAM AND TIM FAMILY MEMBERS

(71) Applicant: THE BRIGHAM AND WOMEN'S HOSPITAL, INC., Boston, MA (US)

(72) Inventors: Richard S. BLUMBERG, Waltham, MA (US); Yu-Hwa HUANG, Boston, MA (US); Yasuyuki KONDO, Boston, MA (US); Amit GANDHI, Billerica, MA (US)

(73) Assignee: THE BRIGHAM AND WOMEN'S HOSPITAL, INC., Boston, MA (US)

(21) Appl. No.: 15/568,633

(22) PCT Filed: Apr. 24, 2015
(86) PCT No.: PCT/US15/27555

§ 371 Date: Oct. 23, 2017

(51) Int. Cl. C07K 16/28; C07K 14/705; A61K 38/17; A61K 39/395; A61P 35/00; A61P 37/06

(52) U.S. Cl. CPC C07K 16/2803; C07K 14/70503; A61K 38/1774; A61K 2039/505; A61P 35/00; A61P 37/06; C07K 2317/76; A61K 39/3955

(57) ABSTRACT

Described herein are compositions and methods for modulating immune response, which can be upregulated or down regulated by enhancement or inhibition of interactions between CEACAM family members and TIM family members.

### GOVERNMENT SUPPORT

This invention was made with government support under Grant No. DK53056, awarded by the National Institutes of Health. The government has certain rights in the invention.

### FIELD OF THE INVENTION

The present invention relates to molecular immunology.

### BACKGROUND

The immune system protects the body from foreign invaders and diseased cells, but immune disorders, particularly those associated with T-cell tolerance, such as cancers, can wreak havoc. According to the most recent data from the World Health Organization, ten million people around the world were diagnosed with cancer in 2000, and six million died from it. Moreover, statistics indicate that the cancer incidence rate is on the rise around the globe. In America, for example, projections suggest that fifty percent of those alive today will be diagnosed with some form of cancer at some point in their lives.

T-cell tolerance is also implicated in immune suppression that can be desirable, for example, in autoimmune diseases and in organ transplant situations, wherein an overactive immune response can cause great permanent damage to the afflicted individual and or donor organ. More specifically, autoimmune disorders are caused by dysfunctional immune responses directed against the body's own tissues, resulting in chronic, multisystem impairments that differ in clinical manifestations, course, and outcome. Autoimmune diseases are on the rise in the U.S. and around the world. In the U.S. alone, some fifty million are affected, and autoimmune disease is one of the top ten causes of death in women under the age of 65, is the second highest cause of chronic illness, and the top cause of morbidity in women.

### SUMMARY

Provided herein are compositions and methods for the modulation of an immune response, such as T-cell tolerance, which can be upregulated or down regulated by concurrent enhancement or inhibition of CEACAM family and TIM family member interactions, also referred to herein as “CEA-TIM interactions.” The compositions and methods described herein are based, in part, on the discovery that not only do CEACAM-1 and TIM-4 physically interact in vivo, but members of the CEACAM family of proteins broadly possess the capacity to bind members of the TIM family. The implications of this varied heterologous binding capacity are many, contributing to or potentially contributing to highly nuanced regulation of processes influenced by these proteins. The interaction of individual CEACAM family members with individual members of the TIM family can have positive or negative effects on immune activities, such as T cell activities, depending upon the amount of each and the presence or absence of other competing factors among other things. The participation of varied CEACAM and TIM family members in immune cell regulation provides a range of targets for modification of immune cell regulations and immune responses. Thus, described herein are agents that specifically target selected pairs of CEACAM and TIM family members to specifically modify their interactions and thereby modulate immune responses.

Provided herein, in some aspects, are compositions for modulating a heterodimeric interaction between a TIM family member and a CEACAM family member comprising a CEA-TIM specific agent that specifically binds to the TIM family member, the CEACAM family member, or both the TIM family member and the CEACAM family member.

In some embodiments of these aspects and all such aspects described herein, the CEA-TIM specific agent binds TIM-4 and/or CEACAM1 and inhibits signaling mediated by the interaction of TIM-4 and CEACAM-1.

In some embodiments of these aspects and all such aspects described herein, the CEA-TIM specific agent binds TIM-4 and/or CEACAM-1 and increases signaling mediated by the interaction of TIM-4 and CEACAM-1.

In some embodiments of these aspects and all such aspects described herein, the CEA-TIM specific agent inhibits signaling mediated by the interaction of K124 of TIM-4 of SEQ ID NO: 2 and N76 of CEACAM1 of SEQ ID NO: 4; K65 of TIM4 of SEQ ID NO: 2 and E132 of CEACAM-1 of SEQ ID NO: 4; G63 of TIM-4 of SEQ ID NO: 2 and E133 of CEACAM1 of SEQ ID NO: 4; or any combination thereof.

In some embodiments of these aspects and all such aspects described herein, the CEA-TIM specific agent increases signaling mediated by the interaction of K124 of TIM-4 of SEQ ID NO: 2 and N76 of CEACAM1 of SEQ ID NO: 4; K65 of TIM4 of SEQ ID NO: 2 and E132 of CEACAM-1 of SEQ ID NO: 4; G63 of TIM-4 of SEQ ID NO: 2 and E133 of CEACAM1 of SEQ ID NO: 4; or any combination thereof.

In some embodiments of these aspects and all such aspects described herein, the CEA-TIM specific agent binds TIM-4 at one or more amino acid residues selected from G63, K65, N101, N121, D122, and K124 of SEQ ID NO: 2.

In some embodiments of these aspects and all such aspects described herein, the CEA-TIM specific agent binds CEACAM-1 at one or more amino acid residues selected from Y68, N76, R77, Q78, G81, Q123, E132, and E133 of SEQ ID NO: 4.

In some embodiments of these aspects and all such aspects described herein, the CEA-TIM specific agent binds TIM-1 and/or CEACAM-1 and inhibits signaling mediated by the interaction of TIM-1 and CEACAM-1.

In some embodiments of these aspects and all such aspects described herein, the CEA-TIM specific agent binds TIM-1 and/or CEACAM-1 and increases signaling mediated by the interaction of TIM-1 and CEACAM-1.

In some embodiments of these aspects and all such aspects described herein, the CEA-TIM specific agent binds TIM-1 at one or more amino acid residues selected from T56, Q58, N94, N114, D115, and K117 of SEQ ID NO: 1.

In some embodiments of these aspects and all such aspects described herein, the CEA-TIM specific agent binds CEACAM-1 at one or more amino acid residues selected from Y68, V73, N76, R77, Q78, G81, Q123, E132, and E133 of SEQ ID NO: 4.

In some embodiments of these aspects and all such aspects described herein, the CEA-TIM specific agent binds TIM-1 and/or CEACAM-3 and inhibits signaling mediated by the interaction of TIM-1 and CEACAM-3.

In some embodiments of these aspects and all such aspects described herein, the CEA-TIM specific agent binds TIM-1 and/or CEACAM-3 and increases signaling mediated by the interaction of TIM-1 and CEACAM-3.

In some embodiments of these aspects and all such aspects described herein, the CEA-TIM specific agent binds TIM-1 at one or more amino acid residues selected from T56, Q58, N94, N114, D115, and K117 of SEQ ID NO: 1.

In some embodiments of these aspects and all such aspects described herein, the CEA-TIM specific agent binds CEACAM-3 at one or more amino acid residues selected from Y68, V73, N76, G81, and Q123 of SEQ ID NO: 5.

In some embodiments of these aspects and all such aspects described herein, the CEA-TIM specific agent binds TIM-3 and/or CEACAM-5 and inhibits signaling mediated by the interaction of TIM-3 and CEACAM-5.

In some embodiments of these aspects and all such aspects described herein, the CEA-TIM specific agent binds TIM-3 and/or CEACAM-5 and increases signaling mediated by the interaction of TIM-3 and CEACAM-5.

In some embodiments of these aspects and all such aspects described herein, the CEA-TIM specific agent binds TIM-3 at one or more amino acid residues selected from P50, C52, C58, E62, C63, G64, R69, N99, T101, C109, C110, R111, N119, D120, and K122 of SEQ ID NO: 3.

In some embodiments of these aspects and all such aspects described herein, the CEA-TIM specific agent binds CEACAM-5 at one or more amino acid residues selected from Y68, V73, N76, Q78, and G81 of SEQ ID NO: 7.

In some embodiments of these aspects and all such aspects described herein, the CEA-TIM specific agent binds TIM-4 and/or CEACAM-6 and inhibits signaling mediated by the interaction of TIM-4 and CEACAM-6.

In some embodiments of these aspects and all such aspects described herein, the CEA-TIM specific agent binds TIM-4 and/or CEACAM-6 and increases signaling mediated by the interaction of TIM-4 and CEACAM-6.

In some embodiments of these aspects and all such aspects described herein, the CEA-TIM specific agent binds TIM-4 at one or more amino acid residues selected from G63, K65, N101, N121, D122, and K124 of SEQ ID NO: 2.

In some embodiments of these aspects and all such aspects described herein, the CEA-TIM specific agent binds CEACAM-6 at one or more amino acid residues selected from Y68, V73, N76, G81, and Q123 of SEQ ID NO: 8.

In some embodiments of these aspects and all such aspects described herein, the CEA-TIM specific agent binds TIM-3 and/or CEACAM-1 and inhibits signaling mediated by the interaction of TIM-3 and CEACAM-1.

In some embodiments of these aspects and all such aspects described herein, the CEA-TIM specific agent binds TIM-3 and/or CEACAM-1 and increases signaling mediated by the interaction of TIM-3 and CEACAM-1.

In some embodiments of these aspects and all such aspects described herein, the CEA-TIM specific agent binds TIM-3 at one or more amino acid residues selected from P50, C52, C58, E62, C63, G64, R69, N99, T101, C109, C110, R111, N119, D120, and K122 of SEQ ID NO: 3.

In some embodiments of these aspects and all such aspects described herein, the CEA-TIM specific agent binds TIM-3 at one or more amino acid residues selected from C52, C63, G64, N99, T101, C109, N119, and K122 of SEQ ID NO: 3.

In some embodiments of these aspects and all such aspects described herein, the CEA-TIM specific agent binds CEACAM-1 at one or more amino acid residues selected from Y68, V73, N76, R77, Q78, G81, Q123, E132, and E133 of SEQ ID NO: 4.

In some embodiments of these aspects and all such aspects described herein, the CEA-TIM specific agent comprises an antibody or antigen binding portion thereof, a peptide or fusion protein, or a small molecule compound.

In some embodiments of these aspects and all such aspects described herein, the CEA-TIM specific agent comprises a soluble TIM family member or a functional fragment or peptide variant thereof.

In some embodiments of these aspects and all such aspects described herein, the CEA-TIM specific agent comprises a soluble CEACAM family member or a functional fragment or peptide variant thereof.

Provided herein, in some aspects, are methods for promoting T-cell tolerance in a subject in need thereof, the method comprising administering a therapeutically effective amount of any of the CEA-TIM specific agents that bind TIM-3 and/or CEACAM-1 and increase signaling mediated by the interaction of TIM-3 and CEACAM-1 described herein.

In some embodiments of these aspects and all such aspects described herein, the subject has an autoimmune disease.

In some embodiments of these aspects and all such aspects described herein, the subject is an organ or transplant recipient.

In some aspects, provided herein are methods for decreasing T-cell tolerance or decreasing T-cell functional exhaustion or increasing an immune response in a subject in need thereof, the method comprising administering a therapeutically effective amount of any of the CEA-TIM specific agents that bind TIM-3 and/or CEACAM-1 and decreases signaling mediated by the interaction of TIM-3 and CEACAM-1 described herein.

In some embodiments of these aspects and all such aspects described herein, the subject has a cancer or a tumor.

In some embodiments of these aspects and all such aspects described herein, the subject has a chronic infection.

Also provided herein, in some aspects are uses of CEA-TIM specific agents that bind TIM-3 and/or CEACAM-1 and increase signaling mediated by the interaction of TIM-3 and CEACAM-1 described herein for promoting T-cell tolerance in a subject in need thereof.

In some embodiments of these aspects and all such aspects described herein, the subject has an autoimmune disease

In some embodiments of these aspects and all such aspects described herein, the subject is an organ or transplant recipient.

Also provided herein, in some aspects are uses of CEA-TIM specific agents that bind TIM-3 and/or CEACAM-1 and decrease signaling mediated by the interaction of TIM-3 and CEACAM-1 described herein for decreasing T-cell tolerance or decreasing T-cell functional exhaustion or increasing an immune response in a subject in need thereof.

In some embodiments of these aspects and all such aspects described herein, the subject has a cancer or a tumor.

In some embodiments of these aspects and all such aspects described herein, the subject has a chronic infection.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the phrases “modulating a heterodimeric interaction between a TIM family member and a CEACAM family member” or “modulating a heterophilic interaction between a TIM family member and a CEACAM family member” refers to increasing or a decreasing an activity or function of a CEACAM family member or a TIM family member, relative to a subject not treated with a CEA-TIM specific agent that modulates the interaction between the CEACAM family member and the TIM family member. As will be clear to the skilled person, “modulating” can also involve effecting a change (which can either be an increase or a decrease) in affinity, avidity, specificity and/or selectivity of a target or antigen, such as a CEACAM family member or a TIM family member, for one or more of its ligands, binding partners, partners for association into a heteromultimeric form, or substrates; and/or effecting a change (which can either be an increase or a decrease) in the sensitivity of the target or antigen, such as a CEACAM family member or a TIM family member, for one or more conditions in the medium or surroundings in which the target or antigen is present (such as pH, ionic strength, the presence of co-factors, etc.), compared to the same conditions but without the presence of a CEA-TIM specific agent. Again, this can be determined in any suitable manner and/or using any suitable assay known per se or described herein, depending on the target involved. “Modulating” can also mean effecting a change (i.e., an activity as an agonist, as an antagonist, or as a reverse agonist, respectively, depending on the target or antigen, such as a CEACAM family member or a TIM family member, and the desired biological or physiological effect) with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay (in vitro, and/or usually cellular or in vivo assay) known per se or described herein, depending on the target or antigen involved.

As used herein, “a CEA-TIM specific agent” refers to a protein-binding agent that specifically binds to a TIM family member, a CEACAM family member, or both a TIM family member and a CEACAM family member, and permits modulation of interactions between a CEACAM family member and a TIM family member, as those terms are defined herein. Such agents include, but are not limited to, antibodies (“antibodies” includes antigen-binding portions of antibodies such as epitope- or antigen-binding peptides, paratopes, functional CDRs; recombinant antibodies; chimeric antibodies; tribodies; midibodies; or antigen-binding derivatives, analogs, variants, portions, or fragments thereof), protein-binding agents, small molecules, recombinant proteins, fusion proteins, peptides, aptamers, avimers, and protein-binding derivatives, portions or active fragments thereof of recombinant proteins, fusion proteins, peptides, aptamers, and avimers. Where a CEA-TIM specific agent acts to inhibit interaction between a CEACAM family member or a TIM family member, it can be referred to as a CEA-TIM inhibitor or antagonist, and, conversely, where a CEA-TIM specific agent acts to increase or enhance or mimic interaction between a CEACAM family member or a TIM family member, it can be referred to as a CEA-TIM agonist or potentiator.

As used herein, “selectively binds” or “specifically binds” refers to the ability of a polypeptide domain described herein to bind to a target, such as a molecule present on the cell-surface, with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if a polypeptide agent described herein binds to TIM-3 with a K_{D} of 10⁻⁵ M or lower, but not to another, unrelated molecule with the same affinity, then the agent is said to specifically bind TIM-3. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

The term “antibody” as used herein, whether in reference to an anti-TIM family member antibody or anti-CEACAM family member antibody, refers to a full length antibody or immunoglobulin, IgG, IgM, IgA, IgD or IgE molecule, or a protein portion thereof that comprises only a portion of an intact antibody, generally including an antigen binding site of the intact antibody and thus retaining the ability to bind a target, such as an epitope or antigen. Antibodies specific for or that selectively bind a TIM family member or a CEACAM family member, whether an activator/agonist antibody or blocking or antagonist antibody, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The term “functional fragment” as used herein refers to a fragment of a TIM family member or CEACAM family member that possesses a biological activity that is substantially similar to a biological activity of the entity or molecule of which it is a fragment, derivative or variant. By “substantially similar” in this context is meant that the biological activity, e.g., ability to bind a desired TIM or CEACAM family member and inhibit or mimic endogenous binding of the desired TIM or CEACAM family member is at least 60% as active as a reference, e.g., a corresponding wild-type TIM or CEACAM family member, and preferably at least 65% as active, at least 75% as active, at least 85% as active, at least 90% as active, 95% as active, at least 100% as active or even higher (i.e., the variant or derivative has greater activity than the wild-type), e.g., 110% as active, 120% as active, or more. Assays to measure the biological activity of a given TIM family member or CEACAM family member functional fragment are known in the art and described herein.

### BRIEF DESCRIPTION OF THE DRAWINGS

FIG. 1 demonstrates co-expression of human CEACAM1 and human TIM-4 in HEK293T cells, where all hTIM-4 are hCEACAM1-positive.

FIG. 2 demonstrates co-expression of CEACAM1 and TIM-4 in colonic infiltrating lamina propria iNKT cells. Representative flow cytometry analyses on the colonic infiltrating iNKT cells the expression of CEACAM1 and TIM-4 under physiological condition. Summary of flow cytometry on the colonic infiltrating iNKT cells and parsing out of the cell sub-populations by the expression of CEACAM1 and TIM-3 (n=2, median shown).

FIG. 3 demonstrates reduced number of iNKT cells in secondary lymphoid organ in a CEACAM1- or TIM-4-deficient environment. Representative flow cytometry analyses on the splenic infiltrating iNKT cells, under physiological conditions, shows that the number of iNKT is reduced in Ceacam1−/− and Tim-4−/− mice.

FIG. 4 demonstrates that CEACAM-1 and/or TIM-4 can rescue in an iNKT-mediated colitis model. Survival curves in Oxazolone-colitis model are shown. Colorectal challenge was performed with 1% oxazolone (OX) or 50% EtOH (EtOH) as a control (DAY0) 5 days after skin painting with 3% oxazolone. (each group n=2; WT and Ceacam1−/−, n=3; Tim-4−/−)

FIG. 5 demonstrates that CEACAM-1 and/or TIM-4 show less severity (body weight loss) in an iNKT-mediated colitis model. Body weight changes in WT, Ceacam1−/− and Tim-4−/− mice in Oxazolone-colitis model are shown.

FIG. 6 demonstrates that TIM-4 deficient mice exhibit excess deletional tolerance in SEB model, and that CEACAM1 is required for deletional tolerance. Bar graph showed the percentage of CD4+Vb8+ T cell in SEB induced tolerance model from WT, Ceacam1−/− (CKO) and Tim4−/− (T4KO) mice which received either PBS control or SEB administration (WT, Ceacam1−/− and Tim4−/− mice).

FIG. 7 shows exemplary flow cytometric data CD4⁺Vb8⁺ tolerogenic T cells in WT, Ceacam1−/− and Tim4−/− mice, which received either PBS control or SEB administration.

FIG. 8 shows alignments of CEACAM family members and conserved residues.

FIG. 9 shows alignments of TIM family members and conserved residues.

FIG. 10 depicts a Table identifying conserved residues amongst CEACAM family members and conserved residues amongst TIM family members.

### DETAILED DESCRIPTION

T-cell tolerance functions, in part, to provide a population of immune system cells that recognize self-major histocompatibility complex (MHC) molecule, but do not recognize self-peptides. Previous studies have shown that both carcinoembryonic antigen related cell adhesion molecule 1 (CEACAM1) and Th1-specific cell surface molecule T-cell Immunoglobulin and Mucin domain-containing molecule-3 (TIM3) are important components of immune regulation. Both molecules have been observed previously to be expressed on activated T-cells, especially after prolonged activation. TIM3 and CEACAM1 form a heterodimeric interaction involving specific interaction sites within the membrane-distal domain of both molecules, and CEACAM1 plays a decisive role in determining whether TIM-3 is an activation or inhibitory molecule (WO 2014/022332, published Feb. 6, 2014, the contents of which are herein incorporated by reference in their entireties).

As demonstrated herein, other CEACAM and TIM family members also unexpectedly interact and these interactions modulate T cell and iNKT cell functions and activity. More specifically, CEACAM 3, 5, and 6 share >83% or five out of the six interacting residues that were previously shown to be critical for CEACAM1 and TIM-3 interactions, and residues N99, N119, D120, and K122 of TIM3 are conserved in human TIM1 and TIM4. As demonstrated herein, CEACAM1 can form heterodimers not only with human TIM-3, but also with human TIM-1 and human TIM-4 in cis- and/or in trans-configurations.

More specifically, as demonstrated herein, N42 of CEACAM1 and K122 of TIM4, E98 of CEACAM1 and K64 of TIM4, and E99 of CEACAM1 and G62 of TIM4 interact. Further, as demonstrated herein, when human embryonic kidney (HEK) cells are transfected with human CEACAM1 and human TIM-4, their expression tracks together in the same cell, as opposed to when CEACAM1 is co-transfected with integrin α5, wherein such tracking does not occur.

As also demonstrated herein, expression of CEACAM1 facilitates the expression of TIM4 in both primary T cells as well as iNKT cells, and this co-expression is functionally important. CEACAM1 and TIM-4 expression was observed on primary mouse invariant natural killer T cells (iNKT), which are T cells that are restricted to CD1d, a nonclassical MHC class I like molecule. As shown herein, CEACAM1 or TIM-4 deficient (knockout or KO) mice have the same effects on iNKT cells, such that deficiency of either molecule leads to decreased iNKT cell levels. Further, as shown herein, CEACAM1-deficient (Ceacam1−/−) or TIM-4-deficient (Tim4−/−) mice are both protected from an iNKT cell mediated colitis in an identical fashion, indicating that loss of CEACAM1 ligation by TIM-4, and vice versa, is responsible for this phenotype. Moreover, as described herein, CEACAM1 and TIM-4 cross-regulate each other, because upregulation of CEACAM1 and increased T cell deletion were observed in the staphylococcal enterotoxin B induced tolerance model in Tim4−/− mice. These data indicate that TIM-4 normally restrains CEACAM1, presumably, without wishing to be bound or limited by theory, intracellularly, given the studies with TIM-3 and CEACAM1, such that when TIM-4 is deleted it leads to increased CEACAM1 expression and T cell deletion.

The observations described herein help to explain the Janus-like or dual functions of human TIM-1 and TIM-4 described in the literature, as recently shown for human TIM-3 (i.e., TIM-3 is activating in the absence of CEACAM1 and inhibitory in the presence of CEACAM1). TIM-1, for example, has been shown to promote T cell activation and therefore inhibit T cell tolerance induction. Mechanistically, TIM-1 has been suggested to operate as a T cell receptor (TCR) signal substitute which allows T cells to bypass the classical TCR signaling pathway which re-routes it towards co-stimulatory driven activation. However, TIM-1 has also been suggested to be involved in immune inhibition, as TIM-1 functions in promoting the regulatory behavior of B cells, another adaptive arm of immunity that is thought to be important in promoting immune tolerance. This implicates an inhibitory role for TIM-1. This duality of TIM-1 function indicates that there is another ligand for TIM-1 (and TIM-4, a ligand for TIM-1), which determines its inhibitory versus activating functions as observed for TIM-3. Further, studies implicate the importance of the membrane distal N-domain in TIM-1 and TIM-4 function, but it has not been previously demonstrated that any of the TIM family members can interact with each other through this domain. The only known protein ligand to interact with a TIM-related N-domain is CEACAM-1, thus supporting the structural predictions described herein that CEACAM-1 and other CEACAM family members are common structural partners for the TIM family members, and interact with multiple TIM family members akin to integrins.

Accordingly, provided herein are compositions and methods comprising CEA-TIM specific agents for modulating heterodimeric interactions between TIM family members and CEACAM family members.

### CEA-TIM Specific Agents

Provided herein, in some aspects, are compositions for modulating a heterodimeric interaction between a TIM family member and a CEACAM family member, the composition comprising a CEA-TIM specific agent that specifically binds to the TIM family member, the CEACAM family member, or both the TIM family member and the CEACAM family member.

### TIM Family Members

Members of the T-cell immunoglobulin and mucin domain-containing proteins or “TIM family of proteins,” “TIM family members,” or “TIM family,” as used herein, refer to a family of type-I cell-surface glycoproteins composed of a signal peptide, an extracellular IgV domain, a mucin-like domain, a transmembrane domain, and an intracellular cytoplasmic tail, and include, in humans, TIM1, TIM3, and TIM4, as defined herein. All members share a conserved sequence homology in the IgV domains. Under normal conditions, TIM family expression is largely confined to restricted sets of lymphoid and myeloid lineage cells and kidney epithelial cells. However, the induction of TIM gene family members is frequently observed on cell types such as stromal cells, endothelial cells and transformed cells in chronic viral infections and cancer, indicating that TIM proteins play a critical role in the regulation of these pathological conditions.

TIM Ig-like domains comprise a deep binding pocket flanked by two hydrophobic loops that can extend into a membrane. The crystal structures of the Ig-like domains of murine TIM-1, TIM-2, TIM-3, and TIM-4 have been determined, revealing that they belong to the immunoglobulin variable (IgV) set. The TIM IgV domains are composed of two antiparallel β-sheets with particularly short β-strands, B, E and D in one sheet (BED β-sheet) and A, G, F, C, C′ and C″ β-strands in the other sheet (GFC β-sheet). All TIM IgV domains contain six conserved Cys residues, and the first and last of these six Cys residues bridge the β-sheets, as in other Ig domains.

The four additional Cys residues found in all TIM family members form two additional disulfide bonds that fix the long CC′ loop folded upwards onto the GFC β-sheet. This folded conformation of the CC′ loop onto the GFC β-sheet is a distinct structural feature of all TIM IgV domains. In homologous IgV domains of other Ig superfamily members, the CC′ loop does not cover the GFC face, and the flat, exposed GFC β-sheet is engaged in intermolecular interactions. The tip of the CC′ loop projects parallel to the FG loop at the top of the IgV domain in mTIM-1, mTIM-3, and mTIM-4, generating a distinctive pocket that is used for recognition of ligands, such as PtdSer. In mTIM-1, mTIM-3, and mTIM-4, interactions of the CC′ loop with two conserved basic residues (Arg and Lys) in the F and G β-strands hold the tip of the CC′ loop upwards and make a pocket by enforcing the distance between the CC′ and FG loops. In mTIM-2, the lack of those interactions leads the CC′ loop to adopt a specific helical conformation and project away from the FG loop.

The unique pocket generated by the CC′ loop and the neighboring FG loop, revealed by high resolution analysis of TIM-4 crystals, contains conserved residues that coordinate with metal ions, such as calcium. This conserved binding pocket has been termed the metal ion-dependent ligand-binding site (MILIBS). The hydrophilic moiety of PtdSer penetrates into the cavity built by the CC′ and FG loops, with its acidic phosphate group coordinating with a metal ion that is linked to oxygens in the two main chains and two side chains of Asn and Asp residues in the FG loop. These Asn and Asp residues are conserved in all TIM proteins binding to PtdSer. The Ser residue of PtdSer fits between the metal ion and the tip of the CC′ loop. The PtdSer amine group has specific interactions with the conserved Asp residue involved in metal ion coordination, whereas the carboxylate of the PtdSer Ser is hydrogen bonded to the Ser residue conserved in the CC′ loop of most TIM IgV domains.

The glycerol and fatty acid moiety that anchor the phospholipid in the lipid bilayer interact with the hydrophobic residues present in the FG loop of the mTIM proteins (Leu-Met in mTIM-3 and Phe-Trp in mTIM-4), as well as with the side chain of the Trp residue in the CC′ loop of mTIM-3. The Phe-Trp residues in the FG loop are conserved in mTIM-1 and human TIM-1 and TIM-4 proteins. Contribution of the BC loop to the membrane binding interaction has been proven by mutagenesis studies of mTIM-1, mTIM-3, and mTIM-4. The BC loop is variable among IgV domains of the TIM family and contains mTIM-3 polymorphic residues.

In some embodiments of the compositions and methods described herein, the TIM family member is TIM-1.

The terms “TIM1” or “TIM-1” (also known as HAVCR, HAVCR-1, KIM-1; or KIM1) refer to the amino acid polypeptide having the amino acid sequence of:

MHPQVVILSLILHLADSVAGSVKVGGEAGPSVTLPCHYSGAVTSMCWNRG
SCSLFTCQNGIVWTNGTHVTYRKDTRYKLLGDLSRRDVSLTIENTAVSDS
GVYCCRVEHRGWFNDMKITVSLEIVPPKVTTTPIVTTVPTVTTVRTSTTV
PTTTTVPTTTVPTTMSIPTTTTVLTTMTVSTTTSVPTTTSIPTTTSVPVT
TTVSTFVPPMPLPRQNHEPVATSPSSPQPAETHPTTLQGAIRREPTSSPL
YSYTTDGNDTVTESSDGLWNNNQTQLFLEHSLLTANTTKGIYAGVCISVL
VLLALLGVIIAKKYFFKKEVQQLSVSFSSLQIKALQNAVEKEVQAEDNIY
IENSLYATD (SEQ ID NO: 1, leader sequence is
underlined),

as described by, e.g., UniPro ID: Q96D42 or RefSeqs NP_001166864.1 or NP_036338.2 or, together with any additional naturally occurring allelic, splice variants, and processed forms thereof. Typically, TIM-1 refers to human TIM-1. The term “TIM-1” can also be used to refer to truncated forms or fragments of the TIM-1 polypeptide. Reference to any such forms or fragments of TIM-1 can be identified in the application, e.g., by “TIM-1 (52-71).” Specific residues of TIM-1 can be referred to as, for example, “TIM-1(56), “amino acid T56 of TIM-1,” or “T56 of TIM-1.”

TIM-1 is a highly polymorphic member of the TIM family, with single nucleotide polymorphisms (SNPs) as well as insertion/deletion variants primarily in the mucin domain in both humans and mice. TIM-1 is mainly expressed on T cells and kidney epithelial cells. Several lines of evidence indicate a dual role for TIM-1 in the regulation of T cell-mediated immunity. TIM-1 can act as a costimulatory molecule for T-cell activation, or transduce a negative signal that leads to the inhibition of T-cell effector function. Several studies have reported multiple roles for TIM-1 in creating immunostimulatory or immunosuppressive environments. For example, treatment with anti-TIM-1 antibodies in the effector phase impedes the development of inflammation, while treatment during the priming phase results in a break in immune tolerance. In addition, TIM-1 on the surface of macrophages acts as a phosphatidylserine receptor that phagocytoses apoptotic cells.

Association analysis of the insertion/deletion variants of TIM1 in human subjects with asthma and allergy (atopic diseases) demonstrated that allelic variation of TIM1 contributed to the risk of atopy, and this association was strongest in individuals with past exposure to hepatitis A virus (HAV). Human TIM1 has been associated not only with allergy and asthma but also with autoimmune diseases, indicating that TIM1 regulates the immune system more globally.

In mice, TIM-1 is expressed on activated but not naive CD4+ T cells. Following differentiation, TIM-1 is preferentially expressed on Th2 cells, while Th1 and Th17 cells express little or no TIM-1. TIM-1 is expressed on mast cells, and at low levels on a subpopulation of B cells, and mRNA for TIM-1 has been shown to be present in invariant natural killer T (iNKT) cells. TIM-1 is expressed on tubular epithelial cells following kidney injury.

The TIM-1 IgV domain has a disulfide-dependent conformation in which the CC loop is folded onto the GFC β strands, resulting in a distinctive cleft formed by the CC and PG loops (Santiago et al, 2007, Immunity 26(3):299-310). The cleft built by the CC and FG loops is a binding site for phosphatidylserine (Kobayashi et al, 2007, Immunity 27(6):927̂10). Antibodies directed to the CC′FG cleft of the TIM-1 IgV domain inhibit TIM-1 binding to phosphatidylserine and dendritic cells and exhibit therapeutic activity in vivo in a humanized mouse model of allergic asthma (Sonar et al, 2010, J. Clin. Invest 120: 2767-81).

TIM-1 expression has been reported on activated CD4 but not CD8 T cells, and TIM-1 cross-linking on naïve T cells leads to rapid T cell activation independent of T cell receptor (TCR) signaling, even though TIM-1 is also recruited to the TCR signaling complex and sustains T cell activation. However, several reports indicate that TIM-1 mainly acts as a co-stimulatory molecule for T cells following TCR cross-linking, particularly after Th2 polarization, rather than in the direct control of T cell activation. TIM-1 crosslinking also increases Th1, Th17, and Th2 responses in vivo, whereas blocking TIM-1 induces the generation of Treg cells and allograft survival in transplant models. TIM-1 is also a marker of murine regulatory B cells, and is expressed by activated B cells where it regulates maturation to plasma cells and antibody production. TIM-1 has been detected on DCs, where it confers pro-inflammatory properties, and on mast cells, where it controls Th2-type cytokine production. TIM-1 also serves as a pattern recognition receptor on invariant natural killer cells (iNKT), mediating cell activation when the iNKT cells bind to PS on the surface of cells undergoing apoptosis. Furthermore, recent reports indicate that TIM-1 is a receptor for Zaire Ebola virus and Lake Victoria Marburg virus on mucosal cells, and targets intracellular proteins for degradation.

Blocking TIM-1 significantly reduced airway inflammation and allergic asthma in mouse models, confirming the role of TIM-1 in atopic-like pathologies. Similarly, anti-TIM-1 antibodies reduced ischemia-reperfusion injury in animal models and ameliorated inflammation-associated damage in mouse models of systemic lupus erythematous (SLE) and experimental glomerulonephritis. Interfering with TIM-1-mediated immune processes also severely curtailed the development of skin hypersensitivity and experimental autoimmune encephalomyelitis (EAE), the mouse model of human multiple sclerosis (MS). In support of the data from animal models, TIM-1 plays a role in human pathologies, particularly atopic diseases such as allergy and asthma. A polymorphism in the TIM-1 gene (insertion 157insMTTTVP) increases susceptibility to hepatitis A virus infection, which is associated with protection against the development of asthma and allergy. Several studies have directly associated polymorphisms in TIM-1 (HAVCR1) with susceptibility to atopic diseases, indicating that specific TIM-1 polymorphisms can affect the development of atopy. TIM-1 is also implicated in the development of other human pathologies, including polymorphisms that control susceptibility to, and the persistence of, hepatitis C virus, HIV, and cerebral malaria infections.

In some embodiments of the compositions and methods described herein, the TIM family member is TIM-4.

The terms “TIM4” or “TIM-4” (also known as TIMD4) refer to the mature amino acid polypeptide having the amino acid sequence of:

MSKEPLILWLMIEFWWLYLTPVTSETVVTEVLGHRVTLPCLYSSWSHNSN
SMCWGKDQCPYSGCKEALIRTDGMRVTSRKSAKYRLQGTIPRGDVSLTIL
NPSESDSGVYCCRIEVPGWFNDVKINVRLNLQRASTTTHRTATTTTRRTT
TTSPTTTRQMTTTPAALPTTVVTTPDLTTGTPLQMTTIAVFTTANTCLSL
TPSTLPEEATGLLTPEPSKEGPILTAESETVLPSDSWSSVESTSADTVLL
TSKASDTAVPEQNKTTKTGQMDGIPMSMKNEMPISQLLMIIAPSLGFVLF
ALFVAFLLRGKLMETYCSQKHTRLDYIGDSKNVLNDVQHGREDEDGLFTL
(SEQ ID NO: 2, leader sequence is underlined),

as described by, e.g., UniPro ID: Q96H15 or RefSeqs NP_001140198.1 and NP_612388.2, together with any additional naturally occurring allelic, splice variants, and processed forms thereof. Typically, TIM-4 refers to human TIM-4. The term “TIM-4” can also be used to refer to truncated forms or fragments of the TIM-4 polypeptide. Reference to any such forms or fragments of TIM-4 can be identified in the application, e.g., by “TIM-4 (59-78).” Specific residues of TIM-4 can be referred to as, for example, “TIM-4(63), “amino acid G63 of TIM-4,” or “G63 of TIM-4.”

TIM-4 was identified in the positional cloning of the TIM family and independently as SMUCKLER (spleen, mucin-containing, knockout of lymphotoxin), a gene selectively downregulated in spleens of LTα- or LTβ-deficient mice. In contrast to TIM-1 and TIM-3, TIM-4 is not expressed on T cells but is expressed exclusively on APCs. In humans, TIM-4 is expressed by tingible-body macrophages located in germinal centers of tonsil and in the white pulp of the spleen. TIM-4 is also expressed on CD169+(MOMA-1) marginal zone metallophilic macrophages in the spleen, and in moderate abundance by CD8α+ and CD8α− subsets of CD11c+ DCs, but not on plasmacytoid DCs. TIM-4 lacks a tyrosine phosphorylation motif in its intracellular domain.

Several studies indicate that TIM-4 can play a role in maintaining systemic tolerance, oral tolerance and prevention of food allergy. TIM-4-mediated engulfment is dependent on the actin cytoskeleton, myosin-II motor proteins, and ATP-dependent cellular processes.

In some embodiments of the compositions and methods described herein, the TIM family member is TIM-3.

The terms “TIM3” or “TIM-3” (also known as HAVCR2) refer to the mature amino acid polypeptide having the amino acid sequence of:

(SEQ ID NO: 3)
MFSHLPFDCVLLLLLLLLTRSSEVEYRAEVGQNAYLPCFYTPAAPGNLV
PVCWGKGACPVFECGNVVLRTDERDVNYWTSRYWLNGDFRKGDVSLTIE
NVTLADSGIYCCRIQIPGIMNDEKFNLKLVIKPAKVTPAPTRQRDFTAA
FPRMLTTRGHGPAETQTLGSLPDINLTQISTLANELRDSRLANDLRDSG
ATIRIGIYIGAGICAGLALALIFGALIFKWYSHSKEKIQNLSLISLANL
PPSGLANAVAEGIRSEENIYTIEENVYEVEEPNEYYCYVSSRQQPSQPL
GCRFAMP,

as described by, e.g., UniPro IDs: Q8TDQ0-1 or Q8TDQ0-2 or RefSeq NP_116171.3, together with any additional naturally occurring allelic, splice variants, and processed forms thereof. The term “TIM-3” can also be used to refer to truncated forms or fragments of the TIM-3 polypeptide. Reference to any such forms or fragments of TIM-4 can be identified in the application, e.g., by “TIM-3 (58-77).” Typically, TIM-3 refers to human TIM-3. Specific residues of TIM-3 can be referred to as, for example, “TIM-3(62), “amino acid E62 of TIM-3,” or “E62 of TIM-3.” For example, the IgV region of TIM3, which comprises amino acids 22-124 of SEQ ID NO: 3, can be referred to as TIM3 (22-124). Exemplary single nucleotide polymorphisms found in the human population include, for example, TIM-3(R111W) and TIM-3(C58R) and TIM-3 (R140L).

Structurally, the TIM3 IgV domain, in both mice and humans, has four noncanonical cysteines that form two unique disulfide bonds, which place the CC′ and FG loops in close proximity. The surface formed by these loops form a binding cleft (FG-CC′ cleft) that is not present in other immunoglobulin superfamily (IgSF) members, and mutagenesis studies demonstrated that this surface contributes to the recognition of a non-galectin-9-ligand(s) that is present on a wide range of primary immune cells (Cao et al., 26 Immunity 311 (2007); Anderson et al., 26 Immunity 273 (2007)). Within the FG-CC′ cleft, Glu62 and Arg111 are critical for galactin-9-independent ligand binding. Substitution of Glu62 did not alter phagocytic activity, whereas substitution of Arg111 completely abrogated the activity. The metal-ion-dependent ligand binding site of TIM3, also important for recognition of apoptotic cells, requires N119 and D120, and to a lesser extent I117 and M118, present in the FG loop of the IgV domain. (Nakayama et al., 113 Blood 3821 (2009)). Thus, although a second heterophilic ligand had been predicted to be involved in TIM3 binding (i.e., to the FG-CC′ cleft), the identity and nature of this interaction remained a mystery until the discoveries described herein.

TIM-3 previously had two known ligands, galectin-9 and phosphatidylserine. Galectin-9 is an S-type lectin with two distinct carbohydrate recognition domains joined by a long flexible linker, and has an enhanced affinity for larger poly-N-acetyllactosamine-containing structures. Galectin-9 does not have a signal sequence and is localized in the cytoplasm. However, it can be secreted and exerts its function by binding to glycoproteins on the target cell surface via their carbohydrate chains (Freeman G J et al., Immunol Rev. 2010 Can; 235(1):172-89).

Galectin-9 is expressed broadly including in immune cells and the epithelium of the gastrointestinal tract. Galectin-9 expression is particularly high in mast cells and also found in T cells, B cells, macrophages, endothelial cells, and fibroblasts. Galectin-9 production can be upregulated by IFN-γ. Galectin-9 has also been reported to exert various biologic functions via interaction with CD44 and IgE. Engagement of TIM-3 by galectin-9 leads to Th1 cell death and a consequent decline in IFN-γ production. When given in vivo, galectin-9 had beneficial effects in several murine disease models, including an EAE model, a mouse model of arthritis, in cardiac and skin allograft transplant models, and contact hypersensitivity and psoriatic models (Freeman G J et al., Immunol Rev. 2010 Can; 235(1):172-89). Residues important for TIM-3 binding to galectin-9 include A44 of TIM-3, D74 of TIM-3, and V100 of TIM-3, which undergo N- and/or O-glycosylation.

Both human and mouse TIM-3 have been shown to be receptors for phosphatidylserine (PtdSer), based on binding studies, mutagenesis, and a co-crystal structure, and it has been shown that TIM-3-expressing cells bound and/or engulfed apoptotic cells expressing PtdSer. Interaction of TIM-3 with PtdSer does not exclude an interaction with galectin-9 as the binding sites have been found to be on opposite sides of the IgV domain. Residues important for TIM-3 binding to PtdSer include P50 of TIM-3, E62 of TIM-3, R69 of TIM-3, R111 of TIM-3, and D120 of TIM-3.

### CEACAM Family Members

Carcinoembryonic antigen-related cell adhesion molecules (“CEACAMs”) or “CEACAM family members,” as used herein, refer to a group of immunoglobulin-related vertebrate glycoproteins that are a subgroup of the CEA family of immunoglobulin-related proteins, and are encoded in the human genome by 12 genes, which cluster on chromosome 19q13.

CEACAMs show distinct expression patterns on different cell types. Some CEACAMs are only expressed in certain epithelial or myeloid cells, others are found in various tissues. Most CEACAMs can be seen as modulators of general cellular processes, such as cell adhesion, differentiation, proliferation, and survival. In addition, CEACAMs are utilized by bacterial pathogens as host receptors on epithelial cells, and bacteria-initiated clustering of CEACAMs can induce robust cellular responses including activation of certain kinases, stimulation of small G proteins, cytoskeletal rearrangements, induction of novel gene expression events, enhanced cell adhesion, and receptor endocytosis, similar to physiological stimulation of CEACAMs.

Cancer is one of the disease states linked to aberrant CEACAM function. In particular, human CEACAM1, CEA, and CEACAM6, which can be found on various epithelial cell types and derived carcinomas, are thought to shape the interaction between tumor cells and their stromal counterparts as well as immune cells. Apart from their utilization as clinical biomarkers and therapeutic targets in melanoma, lung, colorectal, and pancreatic cancers, these epithelial CEACAMs are also implicated in morphogenesis, angiogenesis, cell proliferation, cell motility, apoptosis, and regulation of cell matrix attachment, as well as epithelial cell-cell interaction and cell polarization.

Due to differential splicing, human CEACAM1 occurs in 11 isoforms with the number of extracellular Ig domains ranging from one to four. The major isoforms in human cells are CEACAM1-4 and CEACAM1-3, which possess an extracellular amino-terminal IgV-like domain, followed by three (A1, B, A2) or two (A1, B) IgC2-like domains, respectively. Similarly, in other epithelial CEACAMs, such as CEA or CEACAM6, up to six extracellular IgC2-like domains follow the amino-terminal IgV-like domain. Accordingly, engagement of the extracellular domains of epithelial CEACAMs serves as the primary stimulus for CEACAM-mediated transmembrane signaling. Under physiologic conditions, homophilic interactions between CEACAMs on opposing cells are currently understood to be the major trigger of CEACAM-initiated signaling processes, although CEACAMs can also engage in heterophilic interactions, as described herein for the TIM family.

Trans-oligomerization resulting from homophilic interactions between the amino-terminal IgV-like domains of CEACAMs on neighboring epithelial cells is the basis of CEACAM-mediated cell-cell adhesion. This homophilic type of trans-oligomerization is further supported by the presence of IgC2-like domains. Electron tomography studies of soluble and membrane-attached CEACAM1 ectodomains have not only confirmed the critical role of the IgV-like amino-terminal domain for trans-oligomerisation, but also pointed to additional cis-interactions in the extracellular part of CEACAM1. The extracelluar chain of Ig domains in CEACAM1 appears to be rather flexible, but can be stabilized by cis-interactions between either IgV-like domains or IgC2-like domains of parallel CEACAM1 molecules in the same membrane plane. As a consequence, CEACAMs can occur in different oligomerization states, partially dictated by the occurrence of trans- or cis-interactions between their extracellular domains.

The IgC2 domains of CEACAM1 orthologs from human, cattle, mouse and rat show a higher degree of sequence conservation than the amino-terminal IgV-like domain. The lower sequence conservation in the amino-terminal IgV-like domain compared to the IgC2-like domains has always been interpreted as a sign of positive selection for sequence variation in the amino-terminal domain. However, together with the loss of function upon deletion of the IgC2 domains, the relative conservation of the IgC2 domains of epithelial CEACAMs can reflect conserved functions and therefore evolutionary constraints on this region. Importantly, whereas all isoforms of CEACAM1, CEA, and CEACAM6 encompass at least one IgC2-like extracellular domain, CEACAM3 lacks such an extracellular domain. The absence of an IgC2-like extracellular domain in CEACAM3 correlates well with the mechanistically distinct endocytosis mediated by CEACAM3 in comparison to epithelial CEACAMs.

In some embodiments of the compositions and methods described herein, the CEACAM family member is CEACAM-1.

The terms “CEACAM1” or “CEACAM-1” (also known as BGP or BGP1) refer to the amino acid polypeptide having the amino acid sequence of: MGHLSAPLHRVRVPWQGLLLTASLLTFWNPPTTAQLTTESMPFNVAEGKEVLLLVHNLPQQLF GYSWYKGERVDGNRQIVGYAIGTQQATPGPANSGRETIYPNASLLIQNVTQNDTGFYTLQVIKS DLVNEEATGQFHVYPELPKPSISSNNSNPVEDKDAVAFTCEPETQDTTYLWWINNQSLPVSPRLQ LSNGNRTLTLLSVTRNDTGPYECEIQNPVSANRSDPVTLNVTYGPDTPTISPSDTYYRPGANLSLS CYAASNPPAQYSWLINGTFQQSTQELFIPNITVNNSGSYTCHANNSVTGCNRTTVKTIIVTELSPV VAKPQIKASKTTVTGDKDSVNLTCSTNDTGISIRWFFKNQSLPSSERMKLSQGNTTLSINPVKRE DAGTYWCEVFNPISKNQSDPIMLNVNYNALPQENGLSPGAIAGIVIGVVALVALIAVALACFLHF GKTGRASDQRDLTEHKPSVSNHTQDHSNDPPNKMNEVTYSTLNFEAQQPTQPTSASPSLTATEII YSEVKKQ (SEQ ID NO: 4), as described by, e.g., UniPro IDs P13688-1 to P13688-11, or RefSeqs NP_001020083.1, NP_001171742.1, NP_001171744.1, NP_001171745.1, NP_001192273.1, and NP_001703.2, together with any additional naturally occurring allelic, splice variants, and processed forms thereof. The term “CEACAM-1” can also be used to refer to truncated forms or fragments of the CEACAM-1 polypeptide. Reference to any such forms or fragments of CEACAM-1 can be identified in the application, e.g., by “CEACAM-1(35-142).” Typically, CEACAM-1 refers to human CEACAM-1. Specific residues of CEACAM-1 can be referred to as, for example, “CEACAM-1(68), “amino acid Y68 of CEACAM-1,” or “Y68 of CEACAM-1.”

Clinical evidence shows that high-level CEACAM1 expression on tumors and tumor-infiltrating lymphocytes correlates with poor prognosis and high risk of metastasis. CEACAM1 functions as a regulatory co-receptor for both lymphoid and myeloid cell types, and is constitutively expressed in a wide range of tissues and cell types. Its expression on natural killer (NK) cells and T-cells is, however, mainly induced by cytokines and membrane-activating receptor activation. CEACAM1 consists of a single Ig variable domain-like amino terminus, from one to three Ig constant domain-like regions, and a single membrane-spanning segment followed by either a short (CEACAM1-S) or long (CEACAM1-L) cytoplasmic domain (Hinoda et al., 85 PNAS 6959 (1988)). The N-terminal domain has been shown to facilitate homophilic intercellular binding that influences a broad spectrum of cellular processes related to cellular activation and/or cell cycle progression; and is also targeted by the heterophilic adhesins of viral (murine hepatitis virus) and bacterial (Neisseria gonorrhoeae and N. meningitidis, Moraxella catarrhalis, and Haemophilus influenzae) pathogens, allowing their infection of the diverse array of CEACAM1-expressing human cells and tissues in vivo.

When expressed, CEACAM1 is characterized by significant alternate RNA splicing leading to eleven isoforms in humans and at least four isoforms in mice. These isoforms differ in the length of the cytoplasmic tail and the number of extracellular Ig-like domains and are named accordingly. As noted, the majority of CEACAM1 isoforms possess either a long (CEACAM1-L) CT or a short (CEACAM1-S) cytoplasmic tail. The long cytoplasmic tail (˜72 amino acids in humans) contains two immune-receptor tyrosine-based inhibitory motifs (ITIMs).

CEACAM1 harbors a cytoplasmic domain, which can either be long (L; 71 amino acids in humans) or short (S; 10 amino acids). The “L” isoforms encompass a functional immunoreceptor tyrosine-based inhibitory motif (ITIM) and both CEACAM1-L and CEACAM1-S isoforms are often co-expressed in the same cell, with expression ratios varying between different cell types and between different cellular states. In many cases, expression of the short isoform interferes with CEACAM1-L generated signals. The signal transduction role of CEACAM1 has been mostly attributed to the CEACAM1-L isoform and its cytoplasmic domain. CEACAM1-L can interact with cytoplasmic protein tyrosine kinases and protein tyrosine phosphatases, as well as with calmodulin, β-catenin, actin, filamin, shc, and tropomyosin. Only a few of these interactions are sustained by the short cytoplasmic domain of CEACAM1-4S. In CEACAM1-4S the membrane-proximal phenylalanine F454 or lysine K456 residues, respectively, interact with cytoskeletal components and T457 is phosphorylated.

As described herein, previous studies have determined that CEACAM1 is a ligand for itself (homophilic ligation), and is involved in heterophilic ligation with galectin-3 and selectins. CEACAM1 expression, which is low on resting T-cells, is transcriptionally regulated by ligation of T-cells through the TCR/CD3 complex (signal 1) but negatively regulated when the TCR/CD3 complex is co-engaged by the classical co-stimulatory signal (signal 2) provided by CD28. Given the fact that TCR/CD3 ligation alone (in the absence of costimulation) is a common mechanism for the induction of T-cell tolerance, the strong induction of CEACAM1 by such stimulation may, without wishing to be bound or limited by theory, be part of the tolerogenic program.

Further, in regard to the tertiary structure of CEACAM1, the two major isoforms, CEACAM1-4L and CEACAM1-4S, which differ only in their cytoplasmic domains, have extracellular domains (ectodomains) comprised of four glycosylated Ig domains. CEACAM1-induced cell signaling is regulated by its intercellular homophilic binding at the cell surface, which is mediated by the N-terminal Ig domain (D1) in a reciprocal D1-D1 interaction. The basic structure of the IgV N-terminal domain of CEACAM1 is a tertiary fold of a stacked pair of β-pleated sheets. There are nine component 13 strands, with strands A, B, E, and D lying in one sheet and strands C, C′, C″, F, and G being antiparallel in the other sheet. The GFCC′ face of the N-terminal domain of CEACAM1 is known to be crucial for mediating homophilic adhesion. Homophilic and heterophilic interactions have been observed for other adhesion receptor-ligand pairs; such as of CD2 with CD58; ICAM-1 with ICAM-1, LFA-1, rhinoviruses or Plasmodium falciparum-infected erythrocytes; or cadherins with cadherins (Watt et al., 98 Blood 1469 (2001)). These interactions indicated that the GFCC′ faces of the immunoglobulin family members may have evolved, without wishing to be bound or limited by theory, as a sticky patch to recognize a variety of protein-protein interactions (Springer et al., 6 Ann. Rev. Cell Biol. 359 (1990)).

A peptide region responsible for CEACAM heterophilic interactions, for example with Neisseria Opa proteins, is also on the GFCC′C″ face and overlaps partially with the homophilic binding site. Fedarovich et al., D62 Acta Cryst. 971 (2006). In comparison, binding of a murine CEACAM1 to murine coronavirus requires a uniquely folded CC′ loop, in which murine amino acids 34 to 52 play a crucial role (Tan et al., 21 EMBO J. 2076 (2002); Watt et al., 2001). Additionally, murine amino acids between residues 27 to 42 (particularly D27L28F29) and S32, Y34, V39, Q44, Q89, and 191 on the GFCC′C″ face form differential adhesioptopes for the binding of H. influenzae, and the N. gonorhheae and N. meningiditis Opa proteins. These adhesiotopes are likely a groove; formed by homophilic cis binding that involves murine V39 and D40 CC′ loop residues, or formed after disruption of CEACAM1 cis dimerization by cytokine (e.g., TNFα) activation that precedes CEACAM1/pathogen binding (Watt et al., 2001).

Moreover, the IgC2 domain of CEACAM1 has also been implicated in coronavirus and H. influenzae receptor activity Immobilized CEACAM1, in which the tertiary structure of this highly flexible molecule is limited, also exhibits decreased adhesion, further implicating the cytoplasmic regions of the molecule (e.g., intracellular dimerization) in both homophilic and heterophilic interactions and signaling. Further, the formation of homodimers in cis has been characterized for multiple splice variants (isoforms), even those lacking IgC2 domains.

The lack of intradomain disulfide bridges in the N-terminal D1 domain renders the CEACAM1 ectodomain highly flexible. CEACAM1 exists in the cell membrane in microclusters; whereas homophilic binding triggers reorganization that results in two different kinds of dimers, as well as trimers and higher-order oligomers. Because of the hinge regions between the Ig domains, antiparallel trans-dimers (C-dimers) and parallel cis-dimers (A-dimers) can be formed. The N-terminal D1 domain participates in both C- and A-dimerization, while the D2-D4 domains are involved only in A-dimerization. Divalent cations decreased ectodomain flexibility and enhanced formation of multimeric complexes, which are further implicated in CEACAM1-mediated cell adhesion. Importantly, the dimerization of the ectodomains is transduced by the transmembrane domains to the cytoplasmic domains, thus, in turn, directing intracellular signaling. Another binding site might be implicated across the ABED face, depending on the level and flexibility of glycosylations on the CEACAM1 molecule (Klaile et al., 187 J. Cell Biol. 553 (2009)).

CEACAM1 function is also likely impacted by glycosylation. Carbohydrates account for up to sixty percent of the weight of CEACAM1 expressed on the cell surface. The role of this enormous carbohydrate content allows for the construction of “subspecies” of CEACAM1 isoforms. For example, the ability of CEACAM1 to express sialyl Lewis x modifications can affect leukocyte homing (Chen et al., 86 J. Leuk. Biol. 195 (2009)).

Discriminating between cis and trans interactions in both homophilic and heterophilic adhesions, whether expressed on the same or opposing cells are important, with cis interactions being able to inhibit or enhance interactions in trans. Homophilic interactions or dimerization in cis can thus maintain, create, or obliterate the receptor conformation for heterophilic binding or cell signaling. For example, homophilic interactions or dimerization of CEACAM1 molecules in cis can either maintain the receptor in a conformation incapable of interacting with opposing cells, or place the N-terminal GFCC′C″ face in the correct orientation to increase the avidity of binding to homophilic or heterophilic counter-receptors on the same or opposing cells (as is the case with, for example, ICAM-1, the cadherins, and CEA). Engagement of CEACAM1 homophilically on the same epithelial cell can prevent its interaction in trans and can deliver a negative signal inhibiting epithelial cell proliferation, a regulatory mechanism that is lost when CEACAM1 levels are decreased during epithelial tumor formation. Alternatively, the activation of CEACAM1 molecules on the surface of neutrophils during inflammation can control the presentation of sLex residues to E-selectin ligands on endothelial cells and regulate CD11/CD18 and L-selectin levels. On endothelial and epithelial cells, CEACAM1 activation, perhaps by inducing or inhibiting homophilic interactions or dimerization, can also regulate isoform concentrations on the cell surface, orient the molecules, or increase the avidity of adjacent residues on the GFCC′C″ face of CEACAM1 for Neisserial Opa proteins or H. influenzae.

In some embodiments of the compositions and methods described herein, the CEACAM family member is CEACAM-3.

The terms “CEACAM3” or “CEACAM-3” (also known as CD66D, CGM1) refer to the amino acid polypeptide having the amino acid sequence of: MGPPSASPHRECIPWQGLLLTASLLNFWNPPTTAKLTIESMPLSVAEGKEVLLLVHNLPQHLFGY SWYKGERVDGNSLIVGYVIGTQQATPGAAYSGRETIYTNASLLIQNVTQNDIGFYTLQVIKSDLV NEEATGQFHVYQENAPGLPVGAVAGIVTGVLVGVALVAALVCFLLLAKTGRTSIQRDLKEQQP QALAPGRGPSHS SAFSMSPLSTAQAPLPNPRTAASIYEELLKHDTNIYCRMDHKAEVAS (SEQ ID NO: 5), as described by, e.g., UniPro IDs P40198-1 to P40198-3, together with any additional naturally occurring allelic, splice variants, and processed forms thereof. Typically, CEACAM-3 refers to human CEACAM-3. The term “CEACAM-3” can also be used to refer to truncated forms or fragments of the CEACAM-3 polypeptide. Reference to any such forms or fragments of CEACAM-3 can be identified in the application, e.g., by “CEACAM-3(35-142).” Specific residues of CEACAM-3 can be referred to as, for example, “CEACAM-3(81), “amino acid G81 of CEACAM-3,” or “G81 of CEACAM-3.”

In some embodiments of the compositions and methods described herein, the CEACAM family member is CEACAM-4.

The terms “CEACAM4” or “CEACAM-4” (also known as CGM7) refer to the amino acid polypeptide having the amino acid sequence of: MGPPSAAPRGGHRPWQGLLITASLLTFWHPPTTVQFTIEALPSSAAEGKDVLLLACNISETIQAYY WHKGKTAEGSPLIAGYITDIQANIPGAAYSGRETVYPNGSLLFQNITLEDAGSYTLRTINASYDSD QATGQLHVHQNNVPGLPVGAVAGIVTGVLVGVALVAALVCFLLLSRTGRASIQRDLREQPPPAS TPGHGPSHRSTFSAPLPSPRTATPIYEELLYSDANIYCQIDHKADVVS (SEQ ID NO: 6), as described by, e.g., UniPro ID 075871-1 or RefSeq NP_001808.2, together with any additional naturally occurring allelic, splice variants, and processed forms thereof. Typically, CEACAM-4 refers to human CEACAM-4. The term “CEACAM-4” can also be used to refer to truncated forms or fragments of the CEACAM-4 polypeptide. Reference to any such forms or fragments of CEACAM-4 can be identified in the application, e.g., by “CEACAM-4(36-139).” Specific residues of CEACAM-4 can be referred to as, for example, “CEACAM-4(81), “amino acid G81 of CEACAM-4,” or “G81 of CEACAM-4.”

In some embodiments of the compositions and methods described herein, the CEACAM family member is CEACAM-5.

The terms “CEACAM5” or “CEACAM-5” (also known as CEA) refer to the amino acid polypeptide having the amino acid sequence of: MESPSAPPHRWCIPWQRLLLTASLLTFWNPPTTAKLTIESTPFNVAEGKEVLLLVHNLPQHLFGY SWYKGERVDGNRQIIGYVIGTQQATPGPAYSGREIIYPNASLLIQNIIQNDTGFYTLHVIKSDLVN EEATGQFRVYPELPKPSISSNNSKPVEDKDAVAFTCEPETQDATYLWWVNNQSLPVSPRLQLSN GNRTLTLFNVTRNDTASYKCETQNPVSARRSDSVILNVLYGPDAPTISPLNTSYRSGENLNLSCH AASNPPAQYSWFVNGTFQQSTQELFIPNITVNNSGSYTCQAHNSDTGLNRTTVTTITVYAEPPKP FITSNNSNPVEDEDAVALTCEPEIQNTTYLWWVNNQSLPVSPRLQLSNDNRTLTLLSVTRNDVGP YECGIQNKLSVDHSDPVILNVLYGPDDPTISPSYTYYRPGVNLSLSCHAASNPPAQYSWLIDGNIQ QHTQELFISNITEKNSGLYTCQANNSASGHSRTTVKTITVSAELPKPSISSNNSKPVEDKDAVAFT CEPEAQNTTYLWWVNGQSLPVSPRLQLSNGNRTLTLFNVTRNDARAYVCGIQNSVSANRSDPV TLDVLYGPDTPIISPPDSSYLSGANLNLSCHSASNPSPQYSWRINGIPQQHTQVLFIAKITPNNNGT YACFVSNLATGRNNSIVKSITVSASGTSPGLSAGATVGIMIGVLVGVALI (SEQ ID NO: 7), as described by, e.g., UniPro IDs P06731-1 to P06731-2 or RefSeqs NP_001278413.1 and NP_004354.3, together with any additional naturally occurring allelic, splice variants, and processed forms thereof. Typically, CEACAM-5 refers to human CEACAM-5. The term “CEACAM-5” can also be used to refer to truncated forms or fragments of the CEACAM-5 polypeptide. Reference to any such forms or fragments of CEACAM-5 can be identified in the application, e.g., by “CEACAM-5(35-144).” Specific residues of CEACAM-5 can be referred to as, for example, “CEACAM-5(81), “amino acid G81 of CEACAM-5,” or “G81 of CEACAM-5.”

CEACAM-5 is a cell-surface-bound glycoprotein overexpressed and released by many solid tumors that has an autocrine function in cancer cell survival and differentiation. Soluble CEACAM-5 released by tumors is present in the circulation of patients with cancer, where it is used as a marker for cancer progression. Soluble CEACAM-5 is sufficient to induce proangiogenic endothelial cell behaviors, including adhesion, spreading, proliferation, and migration in vitro and tumor microvascularization in vivo. CEACAM-5 induced activation of endothelial cells was dependent on integrin β-3 signals that activate the focal-adhesion kinase and c-Src kinase and their downstream MAP-ERK kinase/extracellular signal regulated kinase and phosphoinositide 3-kinase/Akt effector pathways.

CEACAM-5 comprises seven immunoglobulin-like (Ig-like) domains, namely N, A1, B1, A2, B2, A3 and B3. CEACAM-5 A1, A2 and A3 domains, on one hand, and B1, B2 and B3 domains, on the other hand, show high sequence homologies, the A domains of human CEACAM-5 presenting from 84 to 87% pairwise sequence similarity, and the B domains from 69 to 80%.

In some embodiments of the compositions and methods described herein, the CEACAM family member is CEACAM-6.

The terms “CEACAM6” or “CEACAM-6” (also known as Non-specific crossreacting antigen) refer to the amino acid polypeptide having the amino acid sequence of: MGPPSAPPCRLHVPWKEVLLTASLLTFWNPPTTAKLTIESTPFNVAEGKEVLLLAHNLPQNRIGY SWYKGERVDGNSLIVGYVIGTQQATPGPAYSGRETIYPNASLLIQNVTQNDTGFYTLQVIKSDLV NEEATGQFHVYPELPKPSISSNNSNPVEDKDAVAFTCEPEVQNTTYLWWVNGQSLPVSPRLQLS NGNMTLTLLSVKRNDAGSYECEIQNPASANRSDPVTLNVLYGPDGPTISPSKANYRPGENLNLSC HAASNPPAQYSWFINGTFQQSTQELFIPNITVNNSGSYMCQAHNSATGLNRTTVTMITVSGSAPV LSAVATVGITIGVLARVALI (SEQ ID NO: 8), as described by, e.g., UniPro ID P40199-1 or RefSeq NP_002474.4, together with any additional naturally occurring allelic, splice variants, and processed forms thereof. Typically, CEACAM-6 refers to human CEACAM-6. The term “CEACAM-6” can also be used to refer to truncated forms or fragments of the CEACAM-6 polypeptide. Reference to any such forms or fragments of CEACAM-6 can be identified in the application, e.g., by “CEACAM-6(35-142).” Specific residues of CEACAM-6 can be referred to as, for example, “CEACAM-6(81), “amino acid G81 of CEACAM-6,” or “G81 of CEACAM-6.”

CEACAM-6 is a glycosylphosphoinositol (GPI)-linked cell surface protein and shares high homology with CEACAM-1, CEACAM-7 and CEACAM-8. CEACAM-6 overexpression leads to morphology change similar to epithelium-messenchymal-transformation (Lewis-Wambi et al, 2009), increased invasivenessness (Lewis-Wambi et al, 2009), increased chemoresistance (Duxbury et al, 2004b) and resistance to anoikis (Ordonez et al, 2000). Suppression of CEACAM-6 gene expression or inhibition of CEACAM-6 function can reverse these effects. Expression of CEACAM-6 protein has been reported in a variety of normal human tissues (Buchegger et al, 1984) including granulocytes; however, CEACAM-6 expression is elevated in many solid tumors such as breast, pancreatic, ovarian, lung and colon cancer (Blumenthal et al, 2007). Additionally, CEACAM-6 over-expression in pancreatic cancer tissues promotes pancreatic cancer cell invasion, metastasis, and angiogenesis, making CEACAM-6 a target for pancreatic cancer therapy.

In some embodiments of the compositions and methods described herein, the CEACAM family member is CEACAM-7.

The terms “CEACAM7” or “CEACAM-7” (also known as Carcinoembryonic antigen CGM2) refer to the amino acid polypeptide having the amino acid sequence of: MGSPSACPYRVCIPWQGLLLTASLLTFWNLPNSAQTNIDVVPFNVAEGKEVLLVVHNESQNLYG YNWYKGERVHANYRIIGYVKNISQENAPGPAHNGRETIYPNGTLLIQNVTHNDAGFYTLHVIKE NLVNEEVTRQFYVFSEPPKPSITSNNFNPVENKDIVVLTCQPETQNTTYLWWVNNQSLLVSPRLL LSTDNRTLVLLSATKNDIGPYECEIQNPVGASRSDPVTLNVRYESVQASSPDLSAGTAVSIMIGVL AGMALI (SEQ ID NO: 9), as described by, e.g., UniPro IDs Q14002-1 to Q14002-2 or RefSeqs NP_001278414.1 or NP_008821.2, together with any additional naturally occurring allelic, splice variants, and processed forms thereof. Typically, CEACAM-7 refers to human CEACAM-7. The term “CEACAM-7” can also be used to refer to truncated forms or fragments of the CEACAM-7 polypeptide. Reference to any such forms or fragments of CEACAM-7 can be identified in the application, e.g., by “CEACAM-7(36-142).” Specific residues of CEACAM-7 can be referred to as, for example, “CEACAM-7(81), “amino acid G81 of CEACAM-7,” or “G81 of CEACAM-7.”

In some embodiments of the compositions and methods described herein, the CEACAM family member is CEACAM-8.

The terms “CEACAM8” or “CEACAM-8” (also known as CD67 antigen, Carcinoembryonic antigen CGM6, or Non-specific cross-reacting antigen NCA-95) refer to the amino acid polypeptide having the amino acid sequence of: MGPISAPSCRWRIPWQGLLLTASLFTFWNPPTTAQLTIEAVPSNAAEGKEVLLLVHNLPQDPRGY NWYKGETVDANRRIIGYVISNQQITPGPAYSNRETIYPNASLLMRNVTRNDTGSYTLQVIKLNLM SEEVTGQFSVHPETPKPSISSNNSNPVEDKDAVAFTCEPETQNTTYLWWVNGQSLPVSPRLQLSN GNRTLTLLSVTRNDVGPYECEIQNPASANFSDPVTLNVLYGPDAPTISPSDTYYHAGVNLNLSCH AASNPPSQYSWSVNGTFQQYTQKLFIPNITTKNSGSYACHTTNSATGRNRTTVRMITVSDALVQ GSSPGLSARATVSIMIGVLARVALII (SEQ ID NO: 10), as described by, e.g., UniPro ID P31997-1 or RefSeq NP_001807.2, together with any additional naturally occurring allelic, splice variants, and processed forms thereof. Typically, CEACAM-8 refers to human CEACAM-8. The term “CEACAM-8” can also be used to refer to truncated forms or fragments of the CEACAM-8 polypeptide. Reference to any such forms or fragments of CEACAM-7 can be identified in the application, e.g., by “CEACAM-8(35-142).” Specific residues of CEACAM-8 can be referred to as, for example, “CEACAM-8(81), “amino acid G81 of CEACAM-8,” or “G81 of CEACAM-8.”

CEACAM-8 is a glycosylphosphatidylinositol-anchored membrane glycoprotein with a molecular weight of around 95 kDa. In addition to the membrane-anchored form, a soluble CEACAM-8 form is released extracellularly after stimulation. Both CEACAM-8 versions comprise an identical amino acid sequence with the exception of a leader sequence labeling the designated membrane-bound CEACAM-8. Enzymes that posttranslationally modify CEACAM8 by adding a GPI anchor to fix the protein to the membrane recognize the leader sequence.

Thus, while both CEACAM and TIM family members have been shown to play indispensable roles in regard to immune regulation and tolerance, the heterodimeric interactions between multiple members of these two families of molecules had not been identified or characterized before the discoveries described herein. Accordingly, there are several aspects to the CEACAM family member and TIM family member heterophilic interactions that are provided by the compositions and methods described herein as avenues for modulating immune responses and T-cell tolerance. Modulating one or more of these heterodimeric interactions between a TIM family member and a CEACAM family member provides approaches, in some aspects, for limiting inhibiting immune responses and/or increasing the development of T-cell tolerance. Increasing suppression of the immune system is highly desirable in, for example, a subject suffering from an autoimmune disorder, or a transplant recipient. In other aspects, modulating one or more of these heterodimeric interactions between a TIM family member and a CEACAM family member provides approaches for enhancing immune responses and/or decreasing the development of T-cell tolerance. Relieving the suppression of the immune system is highly desirable in, for example, a subject suffering from cancer or a subject having a chronic infection.

As used herein, “modulating” or “to modulate” generally means either reducing or inhibiting the activity of, or alternatively increasing the activity of, a target or antigen, such as a CEACAM family member or a TIM family member, as measured using a suitable in vitro, cellular, or in vivo assay. In particular, “modulating” or “to modulate” can mean either reducing or inhibiting the activity of, or alternatively increasing a (relevant or intended) biological activity of, a target or antigen, such as a CEACAM family member or a TIM family member, as measured using a suitable in vitro, cellular or in vivo assay (which will usually depend on the target involved), by at least 5%, at least 10%, at least 25%, at least 50%, at least 60%, at least 70%, at least 80%, at least 90%, or more, inclusive, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, inclusive, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more.

Accordingly, as used herein, the phrases “modulating a heterodimeric interaction between a TIM family member and a CEACAM family member” or “modulating a heterophilic interaction between a TIM family member and a CEACAM family member” refers to increasing or a decreasing an activity or function of a CEACAM family member or a TIM family member, relative to a subject not treated with a CEA-TIM specific agent that modulates the interaction between the CEACAM family member and the TIM family member. As will be clear to the skilled person, “modulating” can also involve effecting a change (which can either be an increase or a decrease) in affinity, avidity, specificity and/or selectivity of a target or antigen, such as a CEACAM family member or a TIM family member, for one or more of its ligands, binding partners, partners for association into a heteromultimeric form, or substrates; and/or effecting a change (which can either be an increase or a decrease) in the sensitivity of the target or antigen, such as a CEACAM family member or a TIM family member, for one or more conditions in the medium or surroundings in which the target or antigen is present (such as pH, ion strength, the presence of co-factors, etc.), compared to the same conditions but without the presence of a CEA-TIM specific agent. Again, this can be determined in any suitable manner and/or using any suitable assay known per se or described herein, depending on the target involved. “Modulating” can also mean effecting a change (i.e., an activity as an agonist, as an antagonist, or as a reverse agonist, respectively, depending on the target or antigen, such as a CEACAM family member or a TIM family member, and the desired biological or physiological effect) with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable (in vitro and usually cellular or in assay) assay known per se or described herein, depending on the target or antigen involved.

Modulating can, for example, also involve allosteric modulation of the target, such as a CEACAM family member or a TIM family member; and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

Thus, for example, a CEACAM family member or a TIM family member activity is “decreased” if one or more signaling activities or downstream read-outs of a CEACAM family member or a TIM family member activity is reduced by a statistically significant amount, such as by at least 10%, at least 20%, at least 30%, at least 40%, at least 50%, at least 60%, at least 70%, at least 80%, at least 90%, at least 95%, at least 97%, at least 98%, or more, up to and including at least 100%, in the presence of an agent or stimulus relative to the absence of such modulation. As will be understood by one of ordinary skill in the art, in some embodiments, if a CEACAM family member or a TIM family member activity is decreased, some downstream read-outs will decrease but others can increase (i.e., activities that are normally suppressed by a CEACAM family member or a TIM family member activity), and the converse would be true in those embodiments where a CEACAM family member or a TIM family member activity is increased.

Conversely, a CEACAM family member or a TIM family member activity is “increased” if one or more signaling activities or downstream read-outs of a CEACAM family member or a TIM family member activity is increased by a statistically significant amount, for example by at least 10%, at least 20%, at least 30%, at least 40%, at least 50%, at least 60%, at least 70%, at least 80%, at least 90%, at least 95%, at least 97%, at least 98%, or more, up to and including at least 100% or more, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more, in the presence of an agent or stimulus, relative to the absence of such agent or stimulus.

As used herein, “a CEA-TIM specific agent” refers to a protein-binding agent that specifically binds to a TIM family member, a CEACAM family member, or both a TIM family member and a CEACAM family member, and permits modulation of interactions between a CEACAM family member and a TIM family member, as those terms are defined herein. Such agents include, but are not limited to, antibodies (“antibodies” includes antigen-binding portions of antibodies such as epitope- or antigen-binding peptides, paratopes, functional CDRs; recombinant antibodies; chimeric antibodies; tribodies; midibodies; or antigen-binding derivatives, analogs, variants, portions, or fragments thereof), protein-binding agents, small molecules, recombinant proteins, fusion proteins, peptides, aptamers, avimers, and protein-binding derivatives, portions or active fragments thereof of recombinant proteins, fusion proteins, peptides, aptamers, and avimers. Where a CEA-TIM specific agent acts to inhibit interaction between a CEACAM family member or a TIM family member, it can be referred to as a CEA-TIM inhibitor or antagonist, and, conversely, where a CEA-TIM specific agent acts to increase or enhance or mimic interaction between a CEACAM family member or a TIM family member, it can be referred to as a CEA-TIM agonist or potentiator.

As used herein, “selectively binds” or “specifically binds” refers to the ability of a polypeptide domain described herein to bind to a target, such as a molecule present on the cell-surface, with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if a polypeptide agent described herein binds to TIM-3 with a K_{D} of 10⁻⁵ M or lower, but not to another, unrelated molecule with the same affinity, then the agent is said to specifically bind TIM-3. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

As used herein, the term “target” refers to a biological molecule (e.g., peptide, polypeptide, protein, lipid, carbohydrate) to which a polypeptide domain which has a binding site can selectively bind. The target can be, for example, an intracellular target (e.g., an intracellular protein target) or a cell surface target (e.g., a membrane protein, a receptor protein).

A “target site” or “ligand interaction site” on the target molecule means a site, epitope, antigenic determinant, part, domain or stretch of amino acid residues on the target that is a site for binding to a ligand, receptor or other binding partner, a catalytic site, a cleavage site, a site for allosteric interaction, a site involved in multimerisation (such as homomerization or heterodimerization) of the target; or any other site, epitope, antigenic determinant, part, domain or stretch of amino acid residues on the target or antigen that is involved in a biological action or mechanism of the target, e.g., a TIM family member or a CEACAM family member. More generally, a “ligand interaction site” can be any site, epitope, antigenic determinant, part, domain or stretch of amino acid residues on a target or antigen to which a binding site of a CEA-TIM specific agent described herein can bind, such that the target (and/or any pathway, interaction, signalling, biological mechanism or biological effect in which the target or antigen is involved), e.g., the interaction between a CEACAM family member and a TIM family member, e.g., CEACAM1 and TIM-4, is modulated.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

In some embodiments of the compositions and methods described herein, the CEA-TIM specific agent is an antibody or antigen-binding fragment thereof.

The term “antibody” as used herein, whether in reference to an anti-TIM family member antibody or anti-CEACAM family member antibody, refers to a full length antibody or immunoglobulin, IgG, IgM, IgA, IgD or IgE molecules, or a protein portion thereof that comprises only a portion of an intact antibody, generally including an antigen binding site of the intact antibody and thus retaining the ability to bind a target, such as an epitope or antigen. Antibodies specific for or that selectively bind a TIM family member or a CEACAM family member, whether an activator/agonist antibody or blocking or antagonist antibody, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Examples of portions of antibodies, antigen-binding fragments, or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al., 8 Protein Eng. 1057 (1995); and U.S. Pat. No. 5,641,870).

The term “specificity” refers to the number of different types of antigens or antigenic determinants to which a particular antibody or antigen-binding portion thereof can bind. The specificity of an antibody or antigen-binding portion thereof can be determined based on affinity and/or avidity. The affinity, represented by the equilibrium constant for the dissociation (K_{D}) of an antigen with an antigen-binding protein is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, a CEA-TIM specific agent as defined herein is said to be “specific for” a first target or antigen compared to another target or antigen when it binds to the first antigen with an affinity (as described above, and suitably expressed, for example as a K_{D} value) that is at least 10×, such as at least 100×, and preferably at least 1000×, and up to 10000× or more better than the affinity with which said amino acid sequence or polypeptide binds to the other target or polypeptide. For example, when a CEA-TIM specific agent is “specific for” a target or antigen compared to another target or antigen, it is directed against said target or antigen, but not directed against the other target or antigen.

Avidity is the measure of the strength of binding between an antigen-binding molecule (such as a CEA-TIM specific agent described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as a CEA-TIM specific agent described herein) will bind to their cognate or specific antigen with a dissociation constant (K_{D} of 10⁻⁵ to 10⁻¹² moles/liter or less, such as 10⁻⁷ to 10⁻¹² moles/liter or less or 10⁻⁸ to 10⁻¹² moles/liter (i.e., with an association constant (K_{A}) of 10⁵ to 10¹² liter/moles or more, and such as 10⁷ to 10¹² liter/moles or 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. A binding site on an antibody or antigen-binding fragment described herein may bind to the desired antigen with an affinity less than 500 nM, less than 200 nM, or less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

In some embodiments of the compositions and methods described herein, the CEA-TIM specific agent is an antibody or antigen-binding fragment thereof that specifically binds TIM-1 and modulates interaction between a CEACAM family member and TIM-1.

In some embodiments of the compositions and methods described herein, the antibody or antigen-binding fragment thereof that modulates interaction between a CEACAM family member and TIM-1 specifically binds TIM-1 at one or more amino acid residues selected from T56, Q58, N94, N114, D115, and K117 of SEQ ID NO: 1.

In some embodiments of the compositions and methods described herein, the antibody or antigen-binding fragment thereof that specifically binds TIM-1 modulates interaction between CEACAM-1 and TIM-1.

In some embodiments of the compositions and methods described herein, the antibody or antigen-binding fragment thereof that specifically binds TIM-1 modulates interaction between CEACAM-3 and TIM-1.

Antibodies or antigen-binding fragments specific for TIM-1 include, for example, those that specifically bind to the extracellular region of TIM-1, such as the IgV domain of TIM-1. Non-limiting examples include commercially available monoclonal anti-human antibodies, such as anti-Hepatitis A Virus Cellular Receptor 1 (HAVCR1) (AA 23-123), anti-Hepatitis A Virus Cellular Receptor 1 (HAVCR1) (AA 1-364), anti-Hepatitis A Virus Cellular Receptor 1 (HAVCR1) (AA 23-122) antibody, anti-Hepatitis A Virus Cellular Receptor 1 (HAVCR1) (Extracellular Domain) antibody, and anti-Hepatitis A Virus Cellular Receptor 1 (HAVCR1) (AA 21-288) antibody. Antibodies that bind to the BED face of human TIM-1 are described, for example, in WO 2013078089, the contents of which are herein incorporated by reference in their entireties.

In some embodiments of the compositions and methods described herein, the CEA-TIM specific agent is an antibody or antigen-binding fragment thereof specific that specifically binds TIM-4 and modulates interaction between a CEACAM family member and TIM-4.

In some embodiments of the compositions and methods described herein, the antibody or antigen-binding fragment thereof that modulates interaction between a CEACAM family member and TIM-4 specifically binds TIM-4 at one or more amino acid residues selected from G63, K65, N101, N121, D122, and K124 of SEQ ID NO: 2.

In some embodiments of the compositions and methods described herein, the antibody or antigen-binding fragment thereof that specifically binds TIM-4 modulates interaction between CEACAM-1 and TIM-4. In some embodiments of the compositions and methods described herein, the antibody or antigen-binding fragment thereof that specifically binds TIM-4 and modulates interaction between CEACAM-1 and TIM-4 modulates the interaction between K124 of SEQ ID NO: 2 and N76 of SEQ ID NO: 4; K65 of SEQ ID NO: 2 and E132 of SEQ ID NO: 4; and G63 of SEQ ID NO: 2 and E133 of SEQ ID NO: 4

In some embodiments of the compositions and methods described herein, the antibody or antigen-binding fragment thereof that specifically binds TIM-4 modulates interaction between CEACAM-6 and TIM-4.

Antibodies or antigen-binding fragments specific for TIM-4 include, for example, those that specifically bind to the extracellular region of TIM-4, such as the IgV domain of TIM-4. Non-limiting examples include commercially available monoclonal anti-human antibodies.

In some embodiments of the compositions and methods described herein, the CEA-TIM specific agent is an antibody or antigen-binding fragment thereof that specifically binds TIM-3 and modulates interaction between a CEACAM family member and TIM-3.

In some embodiments of the compositions and methods described herein, the antibody or antigen-binding fragment thereof that modulates interaction between a CEACAM family member and TIM-3 specifically binds TIM-3 at one or more amino acid residues selected from P50, C52, C58, E62, C63, G64, R69, N99, T101, C109, C110, R111, N119, D120, and K122 of SEQ ID NO: 3.

In some embodiments of the compositions and methods described herein, the antibody or antigen-binding fragment thereof that modulates interaction between a CEACAM family member and TIM-3 specifically binds TIM-3 at one or more amino acid residues selected from C52, C63, G64, N99, T101, C109, N119, and K122 of SEQ ID NO: 3.

In some embodiments of the compositions and methods described herein, the antibody or antigen-binding fragment thereof that specifically binds TIM-3 modulates interaction between CEACAM-1 and TIM-3. In some embodiments of the compositions and methods described herein, the antibody or antigen-binding fragment thereof that specifically binds TIM-3 and modulates interaction between CEACAM-1 and TIM-3 modulates the interaction between E62 and/or D120 of SEQ ID NO: 3 and Q123 of SEQ ID NO: 4; M118 and/or N119 of SEQ ID NO: 3 and Q78 of SEQ ID NO: 4; and/or K122 of SEQ ID NO: 3 and N76 of SEQ ID NO: 4.

In some embodiments of the compositions and methods described herein, the antibody or antigen-binding fragment thereof that specifically binds TIM-3 modulates interaction between CEACAM-5 and TIM-3.

Antibodies or antigen-binding fragments specific for TIM-3 include, for example, those that specifically bind to the extracellular region of TIM-3, such as the IgV domain of TIM-3. Non-limiting examples include antibodies from the hydridomas 7D11, 10G12, and 11G8 (all mouse IgG1) for human TIM-3; monoclonal anti-human TIM-3, Clone #: 344823, isotype rat IgG2A, from R&D SYSTEMS; anti-human TIM3 antibodies from GALPHARMA; and antibodies from the hydridomas 8B.2C12 and 25F.1D6, rat IgG2a, κ (Nakayama et al., 113 Blood 3821 (2000)).

In some embodiments of the compositions and methods described herein, the CEA-TIM specific agent is an antibody or antigen-binding fragment thereof that specifically binds CEACAM-1 and modulates interaction between a TIM family member and CEACAM-1.

In some embodiments of the compositions and methods described herein, the antibody or antigen-binding fragment thereof that modulates interaction between a TIM family member and CEACAM-1 specifically binds CEACAM-1 at one or more amino acid residues selected from Y68, V73, N76, R77, Q78, G81, Q123, E132, and E133 of SEQ ID NO: 4.

In some embodiments of the compositions and methods described herein, the antibody or antigen-binding fragment thereof that specifically binds CEACAM-1 modulates interaction between TIM-1 and CEACAM-1.

In some embodiments of the compositions and methods described herein, the antibody or antigen-binding fragment thereof that specifically binds CEACAM-1 modulates interaction between TIM-3 and CEACAM-1.

In some embodiments of the compositions and methods described herein, the antibody or antigen-binding fragment thereof that specifically binds CEACAM-1 modulates interaction between TIM-4 and CEACAM-1.

Antibodies or antigen-binding fragments specific for CEACAM-1 include, for example, those that specifically bind to the extracellular region of CEACAM-1. Exemplary anti-CEACAM1 monoclonal antibodies useful in the compositions and methods described herein include AgB10, that inhibits CEACAM1-mediated EAE suppression in mice (Fujita et al., 175 Am. J. Pathol. 1116 (2009)); a CEACAM1 antibody (Chen et al., 2004); murine D14HD11 (Yu et al., 281 J. biol. Chem. 39179 (2006)); murine CEACAM1-specific CC1 (Iijima et al., 199 J. Exp. Med. 471 (2004)); mouse anti-human CEACAM1 MRG-1 (Ortenberg 2012); mouse anti-human CEACAM1 N-domain specific antibodies 5F4, 34B1, and 26H7 (IgG1) (Morales et al., 1999) each of which recognizes the N-terminal domain of CEACAM1 (Watt et al., 2001); and 12-I40-4,4/3/17, COL-4, YG-C28F2, D14HD11, B18.7.7, D11-AD11, HEA 81, CLB-gran-10, F34-187, T84.1, B6.2, and B1.1. Monoclonal antibodies 34B1, 26H7, and 5F4 are also discussed in U.S. Patent Pub. No. 2004/0047858, Therapeutic anti-BGP(C-CAM1) antibodies and uses thereof; U.S. Pat. No. 7,132,255; WO 99/52552, and Morales et al., 163 J. Immunol 1363 (1999). Further assessment of specificity was published in Watt et al., 2001, which described that monoclonal antibody 5F4 binds to a domain within the N-Domain of CEACAM1 that is involved in homophilic interactions between the N-Domains of different CEACAM1 molecules.

In some embodiments, humanized or composite anti-CEACAM1 antibodies can be used or generated from monoclonal antibodies generated in non-human hosts. Such antibodies are described, e.g., WO 2013/82366 published Jun. 6, 2013, and the references discussed therein. For example, monoclonal antibodies 34B1, 26H7, and 5F4 have been sequenced and their heavy and light chain variable regions and corresponding CDR regions are known, as described in WO 2013/82366 published Jun. 6, 2013, the contents of which are herein incorporated by reference in their entireties. For example, in some embodiments of the compositions and methods described herein, the CEACAM1-binding antibody or antigen-binding fragment can be constructed to have CDR regions selected from the following: a heavy chain CDR1 consisting of the amino acid residues SSHGMS (SEQ ID NO: 11, 5F4), SFYGMS (SEQ ID NO: 12, 34B1), or SDYYLY (SEQ ID NO: 13, 26H7); a heavy chain CDR2 consisting of the amino acid residues TISSGGTYTYYPDSVKG (SEQ ID NO: 14, 5F4), TFSGGGNYTYYPDSVKG (SEQ ID NO: 15, 34B1) or TISVGGGNTSYPDSVKG (SEQ ID NO: 16, 26H7); a heavy chain CDR3 consisting of the amino acid residues HDFDYDAAWFAY (SEQ ID NO: 17, 5F4), or HGGLPFYAMDY (SEQ ID NO: 18, 34B1), or GLTTGPAWFAY (SEQ ID NO: 19, 26H7); a light chain CDR1 consisting of the amino acid residues SANSSVSYMY (SEQ ID NO: 20, 5F4), SVSSSISSSNLH (SEQ ID NO: 21, 34B1), KSSQSLLNSSNQKNYLA (SEQ ID NO: 22, 26H7), or RASQKISGYLS (SEQ ID NO: 23, 26H7); a light chain CDR2 consisting of the amino acid residues LTSNLAS (SEQ ID NO: 24, 5F4), SVSSSISSSNLH (SEQ ID NO: 25, 34B1), FASTRES (SEQ ID NO: 26, 26H7), or AASTLDS (SEQ ID NO: 27, 26H7); and a light chain CDR3 consisting of the amino acid residues QQWSSNPPT (SEQ ID NO: 28, 5F4), QQWSSHPFT (SEQ ID NO: 29, 34B1), QQHYSTPWT (SEQ ID NO: 30, 26H7) or LQYASSLMYT (SEQ ID NO: 31, 26H7). See also U.S. Patent Pub. No. 2004/0047858, for example.

In some embodiments of these aspects, an antibody or antigen-binding fragment thereof specific for CEACAM-1 has specificity for the same epitope as the monoclonal anti-CEACAM-1 antibody 5F4, described herein, and produced by hybridoma 5F4. In some embodiments of these aspects, an antibody or antigen-binding fragment thereof specific for CEACAM-1 has specificity for the same epitope as the monoclonal anti-CEACAM-1 antibody 26H7, described herein, and produced by hybridoma 26H7. in some embodiments of these aspects, an antibody or antigen-binding fragment thereof specific for CEACAM-1 has specificity for the same epitope as the monoclonal anti-CEACAM-1 antibody 34B 1, described herein, and produced by hybridoma 34B 1.

In some embodiments of these aspects, an antibody or antigen-binding fragment thereof specific for CEACAM-1 is the antibody produced by hybridoma PTA-9974 or has specificity for the same epitope recognized by the antibody produced by hybridoma PTA-9974, as described in U.S. Pat. No. 8,598,322, the contents of which are herein incorporated by reference in their entireties.

In some embodiments of the compositions and methods described herein, the CEA-TIM specific agent is an antibody or antigen-binding fragment thereof that specifically binds CEACAM-3 and modulates interaction between a TIM family member and CEACAM-3.

In some embodiments of the compositions and methods described herein, the antibody or antigen-binding fragment thereof that modulates interaction between a TIM family member and CEACAM-3 specifically binds CEACAM-3 at one or more amino acid residues selected from Y68, V73, N76, G81, and Q123 of SEQ ID NO: 5.

In some embodiments of the compositions and methods described herein, the antibody or antigen-binding fragment thereof that specifically binds CEACAM-3 modulates interaction between TIM-1 and CEACAM-3.

In some embodiments of the compositions and methods described herein, the CEA-TIM specific agent is an antibody or antigen-binding fragment thereof that specifically binds CEACAM-4 and modulates interaction between a TIM family member and CEACAM-4.

In some embodiments of the compositions and methods described herein, the antibody or antigen-binding fragment thereof that modulates interaction between a TIM family member and CEACAM-4 specifically binds CEACAM-4 at amino acid residue G81 of SEQ ID NO: 6.

In some embodiments of the compositions and methods described herein, the CEA-TIM specific agent is an antibody or antigen-binding fragment thereof specific that specifically binds CEACAM-5 and modulates interaction between a TIM family member and CEACAM-5.

In some embodiments of the compositions and methods described herein, the antibody or antigen-binding fragment thereof that modulates interaction between a TIM family member and CEACAM-5 specifically binds CEACAM-5 at one or more amino acid residues selected from Y68, V73, N76, Q78, and G81 of SEQ ID NO: 7.

In some embodiments of the compositions and methods described herein, the antibody or antigen-binding fragment thereof that specifically binds CEACAM-5 modulates interaction between TIM-3 and CEACAM-5.

In some embodiments of these aspects, an antibody or antigen-binding fragment thereof specific for CEACAM-5 is any of the antibodies described in WO 2014079886 A1, the contents of which are herein incorporated by reference in their entireties.

In some embodiments of the compositions and methods described herein, the CEA-TIM specific agent is an antibody or antigen-binding fragment thereof that specifically binds CEACAM-6 and modulates interaction between a TIM family member and CEACAM-6.

In some embodiments of the compositions and methods described herein, the antibody or antigen-binding fragment thereof that modulates interaction between a TIM family member and CEACAM-6 specifically binds CEACAM-6 at one or more amino acid residues selected from Y68, V73, N76, G81, and Q123 of SEQ ID NO: 8.

In some embodiments of the compositions and methods described herein, the antibody or antigen-binding fragment thereof that specifically binds CEACAM-6 modulates interaction between TIM-4 and CEACAM-6.

Monoclonal antibodies against CEACAM6 are available, including anti-CEACAM6 monoclonal 13-1 (Riley et al, 2009), anti-CEACAM6 MAb (Strickland et al 2009), CEACAM6 monoclonal antibody (M02), clone 1G2 (Abnova), CEACAM6 Mouse anti-Human Monoclonal (5F7) (Antibody LifeSpan Biosciences), Human CEACAM6 MAb (Clone 439424) (R & D Systems).

In some embodiments of these aspects, an antibody or antigen-binding fragment thereof specific for CEACAM-6 is any of the antibodies or antibodies produced by the hybridomas described in US 20130272958, the contents of which are herein incorporated by reference in their entireties.

In some embodiments of the compositions and methods described herein, the CEA-TIM specific agent is an antibody or antigen-binding fragment thereof that specifically binds CEACAM-7 and modulates interaction between a TIM family member and CEACAM-7.

In some embodiments of the compositions and methods described herein, the antibody or antigen-binding fragment thereof that modulates interaction between a TIM family member and CEACAM-7 specifically binds CEACAM-7 at one or more amino acid residues selected from Y68, V73, N76, and G81 of SEQ ID NO: 9.

In some embodiments of the compositions and methods described herein, the CEA-TIM specific agent is an antibody or antigen-binding fragment thereof that specifically binds CEACAM-8 and modulates interaction between a TIM family member and CEACAM-8.

In some embodiments of the compositions and methods described herein, the antibody or antigen-binding fragment thereof that modulates interaction between a TIM family member and CEACAM-8 specifically binds CEACAM-8 at one or more amino acid residues selected from Y68, V73, N76, G81, and Q123 of SEQ ID NO: 10.

Monoclonal antibodies against CEACAM8 are available, including mAb 80H3, mAb B13.9, mAb B4-EA4, mAb BIRMA 17C, mAb BL-B7, mAb G10F5, mAb MF25.1, mAb 12-140-5, mAb JML-H16, mAb Kat4c, mAb TET2, mAb YG-C46A8, mAb YG-051B9, mAb C76G4 and/or mAb YG-C94G7, The antibody mAb 80H3 is e.g. commercially available at Immunotech, Marseilles (France), Serotec, abcam and GeneTex. The company abcam also sells the mAb BL-B7. The mAb G10F5 is e.g. provided by BD Pharmingen, BioLegend and StemCell Technologies. The antibody mAb B13.9 is e.g. distributed via immunotools.com, Caltaq and Hoelzel-Biotech. More suppliers for these antibodies as well as the other antibodies mentioned above are known to the skilled artisan.

In some embodiments of these aspects, an antibody or antigen-binding fragment thereof specific for CEACAM-8 is any of the antibodies or antibodies produced by the hybridomas described in U.S. Pat. No. 8,309,091, the contents of which is herein incorporated by reference in its entirety. In some embodiments of these aspects, an antibody or antigen-binding fragment thereof specific for CEACAM-8 is the monoclonal antibody 80113 or has specificity for the same epitope recognized by the monoclonal antibody 80H3, as described in U.S. Pat. No. 8,309,091, the contents of which are herein incorporated by reference in their entireties.

In some embodiments of the compositions and methods described herein, the CEA-TIM specific agent is a blocking antibody or antigen-binding fragment. As used herein, a “blocking” antibody or an “inhibitory antibody” or “antagonist antibody” (or antigen-binding fragment thereof) is one which inhibits or reduces one or more biological activities of the antigen(s) it binds. In certain embodiments, the blocking antibodies or antagonist antibodies or portions thereof described herein completely inhibit the biological activity of the antigen(s).

In some embodiments of the compositions and methods described herein, the CEA-TIM specific agent is an agonist antibody or antigen-binding fragment. As used herein, an “agonist” antibody or an “activating antibody” (or antigen-binding fragments thereof) is one which increases or potentiates one or more biological activities of the antigen(s) it binds. In certain embodiments, the agonist antibodies or antigen-binding fragments thereof, upon binding to their target antigen, mimic the effect of endogenous ligand binding to the target antigen(s).

In some embodiments of the compositions and methods described herein, a CEA-TIM specific agent is a bispecific polypeptide agent that specifically binds to both a TIM family member and a CEACAM family and modulates interaction between a TIM family member and a CEACAM family member.

As used herein, the term “bispecific polypeptide agent” refers to a polypeptide that comprises a first polypeptide domain which has a binding site that has binding specificity for a first target, and a second polypeptide domain which has a binding site that has binding specificity for a second target, i.e., the agent has specificity for two targets, e.g., CEACAM-1 and TIM-4. The first target and the second target are not the same (i.e., are different targets (e.g., proteins)). The different targets can be co-expressed on the same cell or in cis. A bispecific polypeptide agent can bind targets present on a single cell (heterophilic binding in cis), and/or bind one target on one cell and the other on another cell (heterophilic binding in trans). Accordingly, a bispecific polypeptide agent as described herein can selectively and specifically bind to a cell that expresses the first target and the second target, or two different cells. A non-limiting example of a bispecific polypeptide agent is a bispecific antibody construct. Bispecific antibody constructs comprising antigen-binding portions of antibodies specific for two different antigens, e.g., a TIM family member and a CEACAM family member can be constructed by one of skill in the art. Generally, sequences encoding the antigen-binding domain of a first antibody characterized and known to bind a desired epitope on one antigen can be joined, either directly, or through any of a variety of linkers as known to the ordinarily skilled artisan, to sequences encoding the antigen-binding domain of a second antibody characterized and known to bind a desired epitope on a second antigen. Such sequences can be inserted into an appropriate vector and introduced to a cell to produce the bispecific antibody polypeptide by methods known to those of ordinary skill in the art.

In some embodiments of the compositions and methods described herein, the CEA-TIM specific agent is a soluble TIM family member or a functional fragment or peptide variant thereof. In some embodiments of the compositions and methods described herein, the CEA-TIM specific agent is a soluble CEACAM family member or a functional fragment or peptide variant thereof.

As used herein, a functional fragment or variant of a TIM or CEACAM family member refers to a polypeptide having an amino acid sequence that is at least 90%, at least 95%, at least 97%, at least 99% or 100% identical to the sequence of a given TIM or CEACAM family member or portion thereof, and which retains the ability to specifically bind to or interact with a desired TIM or CEACAM family member. As would be understood by one of ordinary skill in the art, such a functional fragment or variant of a TIM or CEACAM family member could act as an inhibitor, by blocking the interaction between endogenous TIM and CEACAM family members, or act as an activator by mimicking the interaction between endogenous TIM and CEACAM family members. Thus, in some embodiments of the compositions and methods described herein, a CEA-TIM specific agent is a peptide mimetic that can functionally replace or recapitulate a TIM family member in activating a CEACAM family member or vice versa.

The term “functional fragment” as used herein refers to a fragment of a TIM family member or CEACAM family member that possesses a biological activity that is substantially similar to a biological activity of the entity or molecule of which it is a fragment, derivative or variant. By “substantially similar” in this context is meant that the biological activity, e.g., ability to bind a desired TIM or CEACAM family member and inhibit or mimic endogenous binding of the desired TIM or CEACAM family member is at least 60% as active as a reference, e.g., a corresponding wild-type TIM or CEACAM family member, and preferably at least 65% as active, at least 75% as active, at least 85% as active, at least 90% as active, 95% as active, at least 100% as active or even higher (i.e., the variant or derivative has greater activity than the wild-type), e.g., 110% as active, 120% as active, or more. Assays to measure the biological activity of a given TIM family member or CEACAM family member functional fragment are known in the art and described herein.

The terms “analog” or “variant” as used herein in reference to a TIM family member or CEACAM family member refers to a polypeptide or nucleic acid that differs from the naturally occurring polypeptide, e.g., CEACAM-1 or TIM-4, or a domain thereof, or nucleic acid encoding such polypeptide, by one or more amino acid or nucleic acid deletions, additions, substitutions or side-chain modifications, yet retains one or more functions or biological activities of the naturally occurring molecule. Amino acid substitutions include alterations in which an amino acid is replaced with a different naturally-occurring or a non-conventional amino acid residue. Such substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). In some embodiments, the TIM family member or CEACAM family member polypeptides provided herein, or used in the methods described herein, can further comprise post-translational modifications. Exemplary post-translational polypeptide modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides can contain non-amino acid elements, such as lipids, poly- or mono-saccharide, and phosphates.

As used herein the term “derivative” refers to a polypeptide that is derived from an endogenous TIM family member or CEACAM family member as described herein, e.g., a functional fragment, and includes peptides which have been chemically modified by techniques such as adding additional side chains, ubiquitination, labeling, pegylation (derivatization with polyethylene glycol, see, e.g., PCT US2007/024067), and insertion, deletion or substitution of amino acid mimetics and/or unnatural amino acids that do not normally occur in the sequence of an endogenous TIM family member or CEACAM family member that is basis of the derivative. For example, in some embodiments, the TIM family member or CEACAM family member derivatives can comprise a label or epitope tag, e.g., a FLAG epitope or a V5 epitope or an HA epitope “Epitope tags” are usually short peptide sequences for which a specific antibody is available. Well known epitope tags for which specific monoclonal antibodies are readily available include FLAG, influenza virus hemagglutinin (HA), and c-myc tags. In some cases, the fusion domains have a protease cleavage site, such as for Factor Xa or Thrombin, which allows the relevant protease to partially digest the fusion polypeptides and thereby liberate the recombinant polypeptides therefrom. The liberated polypeptides can then be isolated from the fusion domain by subsequent chromatographic separation. Another fusion domain well known in the art is green fluorescent protein (GFP) and other fluorescent molecules, such as red fluorescent protein, yellow fluorescent protein etc.; such labeled molecules can also be referred to as derivatives. These fusions are known to those of skill in the art. See, e.g., PCT US2007/024067.

The term “derivative” also encompasses a derivatized polypeptide, such as, for example, a polypeptide modified to contain one or more-chemical moieties other than an amino acid. The chemical moiety can be linked covalently to the peptide, e.g., via an amino terminal amino acid residue, a carboxy terminal amino acid residue, or at an internal amino acid residue. Such modifications include the addition of a protective or capping group on a reactive moiety in the polypeptide, addition of a detectable label, and other changes that do not adversely destroy the activity of the TIM family member or CEACAM family member fusion protein or functional fragment. In some embodiments, a second heterologous polypeptide can be added to a TIM family member or CEACAM family member functional fragment or soluble TIM family member or CEACAM family member to increase in vivo stability, to modulate its biological activity or localization, or to facilitate purification. Exemplary heterologous polypeptides that can be used to generate TIM family or CEACAM family member fusion proteins or functional fragments for use in the compositions and methods described herein include, but are not limited to, polyhistidine (His or 6His tag), Glu-Glu, glutathione S transferase (GST), thioredoxin, polypeptide A, polypeptide G, an immunoglobulin heavy chain constant region (Fc), and maltose binding polypeptide (MBP), which are particularly useful for isolation of the fusion polypeptides by affinity chromatography. For the purpose of affinity purification, relevant matrices for affinity chromatography, such as glutathione-, amylase-, and nickel- or cobalt-conjugated resins are used. In some embodiments, a TIM family member or CEACAM family member derivative contains additional chemical moieties not normally a part of the molecule. Such moieties can improve its solubility, absorption, biological half life, etc. The moieties can alternatively decrease the toxicity of the molecule, or eliminate or attenuate an undesirable side effect of the molecule, etc. Moieties capable of mediating such effects are disclosed, for example, in Remington's Pharmaceutical Sciences, 18th edition, A. R. Gennaro, Ed., MackPubl., Easton, Pa. (1990).

In some embodiments, the CEA-TIM specific agent is a soluble TIM family member. As used herein, a “soluble TIM family member,” refers to any form of a TIM family member, including, in some embodiments, a functional fragment of a TIM family member, that is dissociated from the cell membrane. A soluble TIM family member can be, for example, in some embodiments, a C-terminal truncated form of a full-length TIM family member or a transmembrane-deleted version of a TIM family member. In some embodiments, the soluble TIM family member comprises a single domain of the extracellular region of the TIM family member, i.e., the IgV domain, the mucin-like domain, the BED β-sheet, the GFC β-sheet, and/or the C-C′ loop. In some embodiments, the soluble TIM family member is an alternatively spliced variant of a full-length TIM family member, which includes the IgV domain and intracellular region, but not the mucin domain or transmembrane region. In some embodiments, the soluble TIM family member includes an extracellular region of a TIM family member.

In some embodiments of the aspects described herein, a TIM-1 functional fragment or soluble TIM-1 that can bind a CEACAM family member, such as CEACAM-1, for use in the compositions and methods described herein comprises TIM-1 (52-71) of SEQ ID NO: 1 or CSLFTCQNGIVWTNGTHVTY (SEQ ID NO: 32). In some embodiments of the aspects described herein, a TIM-1 functional fragment or soluble TIM-1 that can bind a CEACAM family member, such as CEACAM-1, for use in the compositions and methods described herein comprises amino acids TIM-1 (52-56) and TIM-1 (105-112) of SEQ ID NO: 1 linked by a SGSG linker or CSLFTSGSGRVEHRGW (SEQ ID NO: 33).

In some embodiments of the aspects described herein, a TIM-4 functional fragment or soluble TIM-4 that can bind a CEACAM family member, such as CEACAM-1, for use in the compositions and methods described herein comprises TIM-4 (58-77) of SEQ ID NO: 2 or CPYSGCKEALIRTDGMRVTS (SEQ ID NO: 34). In some embodiments of the aspects described herein, a TIM-4 functional fragment or soluble TIM-4 that can bind a CEACAM family member, such as CEACAM-1, for use in the compositions and methods described herein comprises amino acids TIM-4 (59-63) and TIM-4 (113-119) of SEQ ID NO: 3 linked by a SGSG linker or CPYSGSGSGRIEVPGW (SEQ ID NO: 35).

In some embodiments of the aspects described herein, a TIM-3 functional fragment or soluble TIM-3 that can bind a CEACAM family member, such as CEACAM-1, for use in the compositions and methods described herein comprises TIM-3 (58-77) of SEQ ID NO: 3 or CPVFECGNVVLRTDERDVNY (SEQ ID NO: 36). In some embodiments of the aspects described herein, a TIM-3 functional fragment or soluble TIM-3 that can bind a CEACAM family member, such as CEACAM-1, for use in the compositions and methods described herein comprises amino acids 58-62 and 111-118 of TIM-3 of SEQ ID NO: 3 linked by a SGSG linker or CPVFESGSGRIQIPGIM (SEQ ID NO: 38). In some embodiments of the aspects described herein, a TIM-3 functional fragment or soluble TIM-3 that can bind a CEACAM family member, such as CEACAM-1, for use in the compositions and methods described herein comprises amino acids 58-62 and 111-119 of TIM-3 of SEQ ID NO: 3 linked by a SGSG linker or CPVFESGSGRIQIPGIMN (SEQ ID NO: 39). In some embodiments of the aspects described herein, a TIM-3 functional fragment or soluble TIM-3 that can bind a CEACAM family member, such as CEACAM-1, for use in the compositions and methods described herein comprises amino acids 58-62 and 111-120 of TIM-3 of SEQ ID NO: 3 linked by a SGSG linker or CPVFESGSGRIQIPGIMND (SEQ ID NO: 40).

TIM-3 functional fragments, soluble TIM-3 molecules, and TIM-3 fusion proteins thereof for use with the compositions and methods described herein can be made according to methods disclosed elsewhere. See, e.g., PCT US2007/024067, filed 15 Nov. 2007, entitled Therapeutic Uses of TIM3 Modulators; Monney et. al., 415 Nature 536 (2002); Anderson et. al., 318 Science 1141 (2007); U.S. Patent Pubs. No. 2004/0005322; No. 2005/0191721, and “CEACAM1 regulates TIM-3-mediated tolerance and exhaustion”, Nature 517, 386-390 (15 Jan. 2015), the contents of each which are herein incorporated by reference in their entireties.

In some embodiments, the CEA-TIM specific agent is a soluble CEACAM family member. As used herein, a “soluble CEACAM family member,” refers to any form of a CEACAM family member, including, in some embodiments, a functional fragment of a CEACAM family member, that is dissociated from the cell membrane. A soluble CEACAM family member can be, for example, in some embodiments, a C-terminal truncated form of a full-length CEACAM family member or a transmembrane-deleted version of a CEACAM family member. In some embodiments, the soluble CEACAM family member comprises one or more domains of the extracellular region of the CEACAM family member, i.e., the IgV-like domain, the IgC2-like domain, the GFCC′C″ face, the GFCC′ face, and/or the CC′ loop. In some embodiments, the soluble CEACAM family member is an alternatively spliced variant of a full-length CEACAM family member, which includes one or more extracellular domains, and in some embodiments, the intracellular region, but not the transmembrane region.

CEACAM-1 functional fragments, soluble CEACAM-1 molecules, and CEACAM-1 fusion proteins thereof for use with the compositions and methods described herein can be made according to methods disclosed elsewhere. CEACAM1-Fc fusion proteins have been described, including, for example, the extracellular portion of CEACAM1 fused to human IgFc in a mammalian expression vector (Markel et al., 110 J. Clin. Invest. 943 (2002)); pCEP4-N-CEACAM1-Fc see Gallagher, 71 J. Virol. 3129 (1997)), contents of each which are herein incorporated by reference in their entireties; as well as commercially available sources of CEACAM1 fusion proteins, as known to one of ordinary skill in the art.

CEACAM-6 functional fragments, soluble CEACAM-6 molecules, and CEACAM-6 fusion proteins thereof for use with the compositions and methods described herein can be made according to methods disclosed elsewhere, for example, those described in U.S. Pat. No. 8,501,192, the contents of which are herein incorporated by references in their entireties; as well as commercially available sources of CEACAM-6 fusion proteins, as known to one of ordinary skill in the art.

CEACAM-8 functional fragments, soluble CEACAM-8 molecules, and CEACAM-8 fusion proteins thereof for use with the compositions and methods described herein can be made according to methods disclosed elsewhere, for example, those described in U.S. Pat. No. 8,501,192, the contents of which are herein incorporated by references in their entireties; as well as commercially available sources of CEACAM-8 fusion proteins, as known to one of ordinary skill in the art.

In some embodiments of the aspects described herein, the CEA-TIM specific agent is a small molecule. The term “small molecule,” in reference to the CEA-TIM specific agents described herein refers to small molecule compounds below about MW 800 g/mol, capable of stimulating or suppressing an immune response in a patient.

In some embodiments of the compositions and methods described herein, the CEA-TIM specific small molecule specifically binds TIM-1 and modulates interaction between a CEACAM family member and TIM-1.

In some embodiments of the compositions and methods described herein, the CEA-TIM specific small molecule that modulates interaction between a CEACAM family member and TIM-1 specifically binds TIM-1 at one or more amino acid residues selected from T56, Q58, N94, N114, D115, and K117 of SEQ ID NO: 1.

In some embodiments of the compositions and methods described herein, the CEA-TIM specific small molecule that specifically binds TIM-1 modulates interaction between CEACAM-1 and TIM-1.

In some embodiments of the compositions and methods described herein, the CEA-TIM specific small molecule that specifically binds TIM-1 modulates interaction between CEACAM-3 and TIM-1.

In some embodiments of the compositions and methods described herein, the CEA-TIM specific small molecule specifically binds TIM-4 and modulates interaction between a CEACAM family member and TIM-4.

In some embodiments of the compositions and methods described herein, the CEA-TIM specific small molecule that modulates interaction between a CEACAM family member and TIM-4 specifically binds TIM-4 at one or more amino acid residues selected from G63, K65, N101, N121, D122, and K124 of SEQ ID NO: 2.

In some embodiments of the compositions and methods described herein, the CEA-TIM specific small molecule that specifically binds TIM-4 modulates interaction between CEACAM-1 and TIM-4. In some embodiments of the compositions and methods described herein, the CEA-TIM specific small molecule that specifically binds TIM-4 and modulates interaction between CEACAM-1 and TIM-4 modulates the interaction between K124 of SEQ ID NO: 2 and N76 of SEQ ID NO: 4; K65 of SEQ ID NO: 2 and E132 of SEQ ID NO: 4; and G63 of SEQ ID NO: 2 and E133 of SEQ ID NO: 4

In some embodiments of the compositions and methods described herein, the CEA-TIM specific small molecule that specifically binds TIM-4 modulates interaction between CEACAM-6 and TIM-4.

In some embodiments of the compositions and methods described herein, the CEA-TIM specific small molecule specifically binds TIM-3 and modulates interaction between a CEACAM family member and TIM-3.

In some embodiments of the compositions and methods described herein, the CEA-TIM specific small molecule that modulates interaction between a CEACAM family member and TIM-3 specifically binds TIM-3 at one or more amino acid residues selected from P50, C52, C58, E62, C63, G64, R69, N99, T101, C109, C110, R111, N119, D120, and K122 of SEQ ID NO: 3.

In some embodiments of the compositions and methods described herein, the CEA-TIM specific small molecule that modulates interaction between a CEACAM family member and TIM-3 specifically binds TIM-3 at one or more amino acid residues selected from C52, C63, G64, N99, T101, C109, N119, and K122 of SEQ ID NO: 3.

In some embodiments of the compositions and methods described herein, the CEA-TIM specific small molecule that specifically binds TIM-3 modulates interaction between CEACAM-1 and TIM-3. In some embodiments of the compositions and methods described herein, the CEA-TIM specific small molecule that specifically binds TIM-3 and modulates interaction between CEACAM-1 and TIM-3 modulates the interaction between E62 and/or D120 of SEQ ID NO: 3 and Q123 of SEQ ID NO: 4; M118 and/or N119 of SEQ ID NO: 3 and Q78 of SEQ ID NO: 4; and/or K122 of SEQ ID NO: 3 and N76 of SEQ ID NO: 4.

In some embodiments of the compositions and methods described herein, the CEA-TIM specific small molecule that specifically binds TIM-3 modulates interaction between CEACAM-5 and TIM-3.

In some embodiments of the compositions and methods described herein, the CEA-TIM specific small molecule specifically binds CEACAM-1 and modulates interaction between a TIM family member and CEACAM-1.

In some embodiments of the compositions and methods described herein, the CEA-TIM specific small molecule that modulates interaction between a TIM family member and CEACAM-1 specifically binds CEACAM-1 at one or more amino acid residues selected from Y68, V73, N76, R77, Q78, G81, Q123, E132, and E133 of SEQ ID NO: 4.

In some embodiments of the compositions and methods described herein, the CEA-TIM specific small molecule that specifically binds CEACAM-1 modulates interaction between TIM-1 and CEACAM-1.

In some embodiments of the compositions and methods described herein, the CEA-TIM specific small molecule that specifically binds CEACAM-1 modulates interaction between TIM-3 and CEACAM-1.

In some embodiments of the compositions and methods described herein, the CEA-TIM specific small molecule that specifically binds CEACAM-1 modulates interaction between TIM-4 and CEACAM-1.

In some embodiments of the compositions and methods described herein, the CEA-TIM specific small molecule specifically binds CEACAM-3 and modulates interaction between a TIM family member and CEACAM-3.

In some embodiments of the compositions and methods described herein, the CEA-TIM specific small molecule that modulates interaction between a TIM family member and CEACAM-3 specifically binds CEACAM-3 at one or more amino acid residues selected from Y68, V73, N76, G81, and Q123 of SEQ ID NO: 5.

In some embodiments of the compositions and methods described herein, the CEA-TIM specific small molecule that specifically binds CEACAM-3 modulates interaction between TIM-1 and CEACAM-3.

In some embodiments of the compositions and methods described herein, the CEA-TIM specific small molecule specifically binds CEACAM-4 and modulates interaction between a TIM family member and CEACAM-4.

In some embodiments of the compositions and methods described herein, the CEA-TIM specific small molecule that modulates interaction between a TIM family member and CEACAM-4 specifically binds CEACAM-4 at amino acid residue G81 of SEQ ID NO: 6.

In some embodiments of the compositions and methods described herein, the CEA-TIM specific small molecule specifically binds CEACAM-5 and modulates interaction between a TIM family member and CEACAM-5.

In some embodiments of the compositions and methods described herein, the CEA-TIM specific small molecule that modulates interaction between a TIM family member and CEACAM-5 specifically binds CEACAM-5 at one or more amino acid residues selected from Y68, V73, N76, Q78, and G81 of SEQ ID NO: 7.

In some embodiments of the compositions and methods described herein, the CEA-TIM specific small molecule that specifically binds CEACAM-5 modulates interaction between TIM-3 and CEACAM-5.

In some embodiments of the compositions and methods described herein, the CEA-TIM specific small molecule specifically binds CEACAM-6 and modulates interaction between a TIM family member and CEACAM-6.

In some embodiments of the compositions and methods described herein, the CEA-TIM specific small molecule that modulates interaction between a TIM family member and CEACAM-6 specifically binds CEACAM-6 at one or more amino acid residues selected from Y68, V73, N76, G81, and Q123 of SEQ ID NO: 8.

In some embodiments of the compositions and methods described herein, the CEA-TIM specific small molecule that specifically binds CEACAM-6 modulates interaction between TIM-4 and CEACAM-6.

In some embodiments of the compositions and methods described herein, the CEA-TIM specific small molecule specifically binds CEACAM-7 and modulates interaction between a TIM family member and CEACAM-7.

In some embodiments of the compositions and methods described herein, the CEA-TIM specific small molecule that modulates interaction between a TIM family member and CEACAM-7 specifically binds CEACAM-7 at one or more amino acid residues selected from Y68, V73, N76, and G81 of SEQ ID NO: 9.

In some embodiments of the compositions and methods described herein the CEA-TIM specific small molecule that specifically binds CEACAM-8 and modulates interaction between a TIM family member and CEACAM-8.

In some embodiments of the compositions and methods described herein, the CEA-TIM specific small molecule that modulates interaction between a TIM family member and CEACAM-8 specifically binds CEACAM-8 at one or more amino acid residues selected from Y68, V73, N76, G81, and Q123 of SEQ ID NO: 10.

Binding assays can be used to screen for or detect agents that bind to a TIM family member or CEACAM family member, or disrupt the interaction between a TIM family member or CEACAM family member. Because TIM family members and CEACAM family members are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners.

Further, agents that inhibit or enhance the interaction between a TIM family member or CEACAM family member for use in the compositions and methods described herein, including peptide-mimetics or functional fragments of a TIM family member or CEACAM family member, can be identified by, for example, transfecting cells with expression vectors encoding a particular TIM family member or CEACAM family member (e.g., 293T-cells transfected with human CEACAM-1 and TIM-4 expression constructs); contacting the cells with a potential CEA-TIM specific agent; lysing the cells; and characterizing the CECAM-1/TIM-4 interaction in comparison with cells not contacted with agent. Cells can be characterized using, for example, co-immunoprecipitation. An in vitro assay, such as an ELISA assay with plate-bound TIM family member or CEACAM family member fusion proteins can be used and binding of a given ligand monitored, respectively, in the presence of potential CEA-TIM specific agents that can modulate these interactions. Similar studies can be performed with cells transfected with TIM family member or CEACAM family member using CEA-TIM specific agents which mimic or compete for binding of an endogenous ligand to a TIM family member or CEACAM family member respectively. Alternatively or additionally, the use of affinity biosensor methods can be used for binding and competition analyses. Such methods can be based on the piezoelectric effect, electrochemistry, or optical methods, such as ellipsometry, optical wave guidance, and surface plasmon resonance (SPR). Detection methods useful in such assays also include antibody-based methods (i.e., an antibody directed against the “free” protein), direct detection of a reporter moiety incorporated into the “free” protein (such as a fluorescent label), and proximity energy transfer methods (such as a radioactive “free” protein resulting in fluorescence or scintillation of molecules incorporated into the immobilized protein or the solid support).

### Methods of Inhibiting and Increasing Immune Response Using CEA-TIM Specific Agents

By modulating the interactions between a TIM family member and a CEACAM family member, the CEA-TIM specific agents described herein are useful in methods of increasing/enhancing or decreasing/suppressing an immune response, such as decreasing or increasing T cell tolerance, in subjects in need thereof. Modulation of immune responses by CEA-TIM specific agents is useful for specifically enhancing or suppressing an immune response in vivo, which can be useful for the treatment of conditions related to immune function including autoimmune disease, cancer, and transplantation (e.g., bone marrow or organs). Modulation of immune responses, such as T cell tolerance, also is useful in in vitro or non-therapeutic applications including determining whether T-cells of a subject are functional (e.g., proliferation and/or cytotoxic functions), to determine if a treatment has rendered T-cells non-functional, in experimental models of cancer, autoimmune disease, and transplantation, e.g., to determine the effects of increases or decreases in T-cell function on particular organs or physiological processes, and to test for agents which increase or decrease T-cell activity. Other uses will be apparent to one of ordinary skill in the art. The CEA-TIM specific agents that modulate interactions between a TIM family member and a CEACAM family member can be used alone as a primary therapy or in combination with other therapeutics as a combination therapy to enhance the therapeutic benefits of other medical treatments.

Accordingly, provided herein, in some aspects, are methods for modulating an immune response in a subject in need thereof by modulating a heterodimeric interaction between a TIM family member and a CEACAM family member, comprising administering a therapeutically effective amount of a CEA-TIM specific agent that specifically binds to a TIM family member, a CEACAM family member, or both a TIM family member and a CEACAM family member.

As used herein, an “immune response” being modulated by the CEA-TIM specific agents described herein refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, or any combination thereof, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments of the compositions and methods described herein, an immune response being modulated is T-cell tolerance. In some embodiments of the compositions and methods described herein, an immune response being modulated is functional exhaustion, such as T cell functional exhaustion.

As used herein, the terms “increases/promotes T cell tolerance” or “decreases/inhibits T cell tolerance” means that a given treatment or set of conditions leads to a decreased immune response/increased T cell tolerance or increased immune response/decreased T cell tolerance, respectively. One of ordinary skill in the art can determine whether increased or decreased T cell tolerance is achieved using, for example, an SEB model where neonatal injection of staphylococcal enterotoxin B (SEB) induces tolerance in T cells that express reactive T cell receptor (TCR) V beta regions. If T cell tolerance is abrogated or reduced, such as, for example, in the absence of TIM-3 signaling or TIM-3 interactions with a CEACAM family member, then T cells expressing Vbeta8 would have reduced tolerance, i.e., have greater activity. Such a model can be adapted to examine CEA-TIM specific agents using T cell tolerance as the readout. Other models or systems to identify whether a CEA-TIM specific agent promotes or inhibits T cell tolerance include OVA antigen-specific T cell tolerance models using OTII transgenic animals injected with OVA peptide at day 0 and day 4, followed by ex vivo stimulation of lymphocytes with various concentrations of OVA peptide.

As used herein, “unresponsiveness” or “functional exhaustion” with regard to immune cells includes refractivity of immune cells, such as T cells, to stimulation, such as stimulation via an activating receptor or a cytokine. Unresponsiveness can occur, for example, because of exposure to immunosuppressants, exposure to high or constant doses of antigen, or through the activity of inhibitory receptors, such as TIM-3. As used herein, the term “unresponsiveness” includes refractivity to activating receptor-mediated stimulation. Such refractivity is generally antigen-specific and persists after exposure to the antigen has ceased. Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type.

In some embodiments of the methods described herein, the subject being administered a CEA-TIM specific agent for modulating heterodimeric interactions between a TIM family member and a CEACAM family member is in need of increased T cell tolerance, such as a subject having or diagnosed with an autoimmune disease. Accordingly, provided herein, in some aspects, are methods of treating a subject having or diagnosed with an autoimmune disorder by increasing T cell tolerance comprising administering an effective amount of a CEA-TIM specific agent. Also provided herein, in some aspects, are CEA-TIM specific agents for use in increasing T cell tolerance or treating an autoimmune disorder in a subject.

In some embodiments of these methods and uses and all such methods and uses for increasing T cell tolerance or treating an autoimmune disorder described herein, the CEA-TIM specific agent increases interaction between TIM-3 and CEACAM-1.

In some embodiments of these methods and uses and all such methods and uses for increasing T cell tolerance or treating an autoimmune disorder described herein, the CEA-TIM specific agent increases interaction between TIM-3 and CEACAM-5.

In some embodiments of these methods and uses and all such methods and uses for increasing T cell tolerance or treating an autoimmune disorder described herein, the CEA-TIM specific agent decreases interaction between TIM-1 and CEACAM-1.

In some embodiments of these methods and uses and all such methods and uses for increasing T cell tolerance or treating an autoimmune disorder described herein, the CEA-TIM specific agent decreases interaction between TIM-1 and CEACAM-3.

In some embodiments of these methods and uses and all such methods and uses for increasing T cell tolerance or treating an autoimmune disorder described herein, the CEA-TIM specific agent decreases interaction between TIM-4 and CEACAM-1.

In some embodiments of these methods and uses and all such methods and uses for increasing T cell tolerance or treating an autoimmune disorder described herein, the CEA-TIM specific agent decreases interaction between TIM-4 and CEACAM-6.

As used herein, “autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include cancer cells.

The autoimmune diseases to be treated or prevented using the methods described herein, include, but are not limited to: rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Due, at least in part, to the involvement of inflammation in their pathology, autoimmune disease has been recognized also to encompass atherosclerosis and Alzheimer's disease. In one embodiment of the aspects described herein, the autoimmune disease is selected from the group consisting of multiple sclerosis, type-I diabetes, Hashinoto's thyroiditis, Crohn's disease, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments of the methods described herein, the subject being administered a CEA-TIM specific agent for modulating heterodimeric interactions between a TIM family member and a CEACAM family member is in need of increased T cell tolerance, such as an organ or transplant recipient. Accordingly, provided herein, in some aspects, are methods of treating a subject having or receiving an organ or transplant by increasing T cell tolerance comprising administering an effective amount of a CEA-TIM specific agent. Also provided herein, in some aspects, are CEA-TIM specific agents for use in treating an organ or transplant recipient. In some embodiments, the methods described herein are used for increasing transplantation tolerance in a subject, comprising administering to the subject a therapeutically effective amount of a CEA-TIM specific agent; or contacting a cell population with a CEA-TIM specific agent, such as T cell population. Also provided herein, in some aspects, are CEA-TIM specific agents for use in increasing T cell tolerance or increasing transplantation tolerance in a subject.

In some embodiments of these methods and uses and all such methods and uses for increasing T cell tolerance or treating an organ or transplant recipient described herein, the CEA-TIM specific agent increases interaction between TIM-3 and CEACAM-1.

In some embodiments of these methods and uses and all such methods and uses for increasing T cell tolerance or treating an organ or transplant recipient described herein, the CEA-TIM specific agent increases interaction between TIM-3 and CEACAM-5.

In some embodiments of these methods and uses and all such methods and uses for increasing T cell tolerance or treating an organ or transplant recipient described herein, the CEA-TIM specific agent decreases interaction between TIM-1 and CEACAM-1.

In some embodiments of these methods and uses and all such methods and uses for increasing T cell tolerance or treating an organ or transplant recipient described herein, the CEA-TIM specific agent decreases interaction between TIM-1 and CEACAM-3.

In some embodiments of these methods and uses and all such methods and uses for increasing T cell tolerance or treating an organ or transplant recipient described herein, the CEA-TIM specific agent decreases interaction between TIM-4 and CEACAM-1.

In some embodiments of these methods and uses and all such methods and uses for increasing T cell tolerance or treating an organ or transplant recipient described herein, the CEA-TIM specific agent decreases interaction between TIM-4 and CEACAM-6.

“Transplantation tolerance,” as used herein, refers to a lack of rejection of the donor organ by the recipient's immune system. In some such embodiments, the subject is a recipient of an allogenic transplant. The transplant can be any organ or tissue transplant, including but not limited to heart, kidney, liver, skin, pancreas, bone marrow, skin or cartilage.

In some embodiments of the methods described herein, the subject being administered a CEA-TIM specific agent for modulating heterodimeric interactions between a TIM family member and a CEACAM family member is in need of an increased immune response or decreased T cell tolerance or decreased functional exhaustion, such as a subject having or diagnosed with cancer. Accordingly, provided herein, in some aspects, are methods of treating a subject having or diagnosed with cancer by decreasing T cell tolerance or reversing functional exhaustion comprising administering an effective amount of a CEA-TIM specific agent. Also provided herein, in some aspects, are CEA-TIM specific agents for use in decreasing T cell tolerance or treating a cancer or tumor in a subject.

In some embodiments of these methods and uses and all such methods and uses for decreasing T cell tolerance or decreasing T cell functional exhaustion or increasing immune responses for treating a cancer or a tumor in a subject described herein, the CEA-TIM specific agent decreases interaction between TIM-3 and CEACAM-1.

In some embodiments of these methods and uses and all such methods and uses for decreasing T cell tolerance or decreasing T cell functional exhaustion or increasing immune responses for treating a cancer or a tumor in a subject described herein, the CEA-TIM specific agent decreases interaction between TIM-3 and CEACAM-5.

In some embodiments of these methods and uses and all such methods and uses for decreasing T cell tolerance or decreasing T cell functional exhaustion or increasing immune responses for treating a cancer or a tumor in a subject described herein, the CEA-TIM specific agent increases interaction between TIM-1 and CEACAM-1.

In some embodiments of these methods and uses and all such methods and uses for decreasing T cell tolerance or decreasing T cell functional exhaustion or increasing immune responses for treating a cancer or a tumor in a subject described herein, the CEA-TIM specific agent increases interaction between TIM-1 and CEACAM-3.

In some embodiments of these methods and uses and all such methods and uses for decreasing T cell tolerance or decreasing T cell functional exhaustion or increasing immune responses for treating a cancer or a tumor in a subject described herein, the CEA-TIM specific agent increases interaction between TIM-4 and CEACAM-1.

In some embodiments of these methods and uses and all such methods and uses for decreasing T cell tolerance or decreasing T cell functional exhaustion or increasing immune responses for treating a cancer or a tumor in a subject described herein, the CEA-TIM specific agent increases between TIM-4 and CEACAM-6.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments described herein, the methods further comprise administering a tumor or cancer antigen to a subject being administered a CEA-TIM specific agent described herein.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. For example, viral proteins such as hepatitis B (HBV), Epstein-Barr (EBV), and human papilloma (HPV) have been shown to be important in the development of hepatocellular carcinoma, lymphoma, and cervical cancer, respectively. However, tumors use or benefit from a range of different immune evasion mechanisms, such that the immune systems of cancer patients often fail to respond to the tumor antigens. Some examples of cancer antigens that are normally associated with spermatocytes or spermatogonia of the testis, placenta, and ovary include the cancer-testis (CT) antigens BAGE, GAGE, MAGE-1 and MAGE-3, NY-ESO-1, SSX. These antigens are found in melanoma, lymphoma, lung, bladder, colon, and breast carcinomas (e.g., as described in Butterfield et al., J. Immunotherapy 2008; 31:294-309; Markowicz et al., J Clin Oncol 27:15s, 2009 (suppl; abstr 9039)). Cancer antigens normally found in melanocytes, epithelial tissues, prostate, and colon also include the differentiation antigens Gp100, Melan-A/Mart-1, Tyrosinase, PSA, CEA, and Mammaglobin-A. These antigens are found in melanoma, prostate cancer, and in colon and breast carcinomas. Some cancer antigens are shared antigens that are ubiquitously expressed at low levels but overexpressed in cancers. Examples of overexpressed cancer antigens include p53, HER-2/neu, livin, and survivin, found in esophagus, liver, pancreas, colon, breast, ovary, bladder, and prostate carcinomas. Other cancer antigens are unique, such as β-catenin-m, β-Actin/4/m, Myosin/m, HSP70-2/m, and HLA-A2-R170J, which are associated with one or more of melanoma, non-small cell lung cancer, and renal cancer. Still other cancer antigens are the tumor-associated carbohydrate antigens that are normally found in epithelial tissues such as renal, intestinal, and colorectal tissues. These cancer antigens include GM2, GD2, GD3, MUC-1, sTn, abd globo-H, which can be found in melanoma, neuroblastoma, colorectal, lung, breast, ovarian, and prostate cancers. Additional tumor antigens, peptide epitopes, and descriptions thereof are described in U.S. Pat. Nos. 7,906,620; 7,910,692; 8,097,242; 7,935,531; 8,012,468; 8,097,256; 8,003,773; Tartour et al, Immunol Lett 2000; 74(1): 1-3, the contents of which are herein incorporated by reference in their entireties. In some embodiments, the intact cancer antigen is used, whereas in other embodiments, a peptide epitope of the cancer antigen (prepared either by proteolytic digestion or recombinantly) is used.

In some embodiments of the methods described herein, the methods further comprise administering an anti-cancer therapy or agent to the subject being administered a CEA-TIM specific agent.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets PD1, PDL1, PDL2, TIM3 or any TIM family member, CEACAM1 or any CEACAM family member, ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In some embodiments, a chemotherapeutic agent is an agent of use in treating neoplasms such as solid tumors. In some embodiments, a chemotherapeutic agent is a radioactive molecule. One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the symptoms of the disorder being treated, the presence or size of metastases or micrometastases, the size of the primary tumor, and/or the presence or the size of the dormant tumor.

In some embodiments of the methods described herein, the subject being administered a CEA-TIM specific agent for modulating heterodimeric interactions between a TIM family member and a CEACAM family member is in need of an increased immune response or decreased T cell tolerance or decreased functional exhaustion, such as a subject having or diagnosed with an infection, such as a chronic infection. Accordingly, provided herein, in some aspects, are methods of treating a subject having or diagnosed with an infection by decreasing T cell tolerance or reversing functional exhaustion comprising administering an effective amount of a CEA-TIM specific agent. Also provided herein, in some aspects, are CEA-TIM specific agents for use in decreasing T cell tolerance or treating an infection in a subject.

In some embodiments of these methods and uses and all such methods and uses for decreasing T cell tolerance or decreasing T cell functional exhaustion or increasing immune responses for treating an infection in a subject described herein, the CEA-TIM specific agent decreases interaction between TIM-3 and CEACAM-1.

In some embodiments of these methods and uses and all such methods and uses for decreasing T cell tolerance or decreasing T cell functional exhaustion or increasing immune responses for treating an infection in a subject described herein, the CEA-TIM specific agent decreases interaction between TIM-3 and CEACAM-5.

In some embodiments of these methods and uses and all such methods and uses for decreasing T cell tolerance or decreasing T cell functional exhaustion or increasing immune responses for treating an infection in a subject described herein, the CEA-TIM specific agent increases interaction between TIM-1 and CEACAM-1.

In some embodiments of these methods and uses and all such methods and uses for decreasing T cell tolerance or decreasing T cell functional exhaustion or increasing immune responses for treating an infection in a subject described herein, the CEA-TIM specific agent increases interaction between TIM-1 and CEACAM-3.

In some embodiments of these methods and uses and all such methods and uses for decreasing T cell tolerance or decreasing T cell functional exhaustion or increasing immune responses for treating an infection in a subject described herein, the CEA-TIM specific agent increases interaction between TIM-4 and CEACAM-1.

In some embodiments of these methods and uses and all such methods and uses for decreasing T cell tolerance or decreasing T cell functional exhaustion or increasing immune responses for treating an infection in a subject described herein, the CEA-TIM specific agent increases between TIM-4 and CEACAM-6.

In some embodiments of the methods described herein, the subject being administered a CEA-TIM specific agent has an infection with a pathogen, such as a bacterium, virus, fungus, or parasite. In some embodiments of these aspects and all such aspects described herein, the subject has a chronic or persistent infection.

“Persistent infections” refer to those infections that, in contrast to acute infections, are not effectively cleared by the induction of a host immune response. During such persistent infections, the infectious agent and the immune response reach equilibrium such that the infected subject remains infectious over a long period of time without necessarily expressing symptoms. Persistent infections often involve stages of both silent and productive infection without rapidly killing or even producing excessive damage of the host cells. Persistent infections include for example, latent, chronic and slow infections. Persistent infection occurs with viruses including, but not limited to, human T-Cell leukemia viruses, Epstein-Barr virus, cytomegalovirus, herpesviruses, varicella-zoster virus, measles, papovaviruses, prions, hepatitis viruses, adenoviruses, parvoviruses and papillomaviruses.

In a “chronic infection,” the infectious agent can be detected in the subject at all times. However, the signs and symptoms of the disease can be present or absent for an extended period of time. Non-limiting examples of chronic infection include hepatitis B (caused by hepatitis B virus (HBV)) and hepatitis C (caused by hepatitis C virus (HCV)) adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus II. Parasitic persistent infections can arise as a result of infection by, for example, Leishmania, Toxoplasma, Trypanosoma, Plasmodium, Schistosoma, and Encephalitozoon.

In a “latent infection,” the infectious agent (such as a virus) is seemingly inactive and dormant such that the subject does not always exhibit signs or symptoms. In a latent viral infection, the virus remains in equilibrium with the host for long periods of time before symptoms again appear; however, the actual viruses cannot typically be detected until reactivation of the disease occurs. Non-limiting examples of latent infections include infections caused by herpes simplex virus (HSV)-1 (fever blisters), HSV-2 (genital herpes), and varicella zoster virus VZV (chickenpox-shingles).

In a “slow infection,” the infectious agents gradually increase in number over a very long period of time during which no significant signs or symptoms are observed. Non-limiting examples of slow infections include AIDS (caused by HIV-1 and HIV-2), lentiviruses that cause tumors in animals, and prions.

Examples of infectious viruses include: Retroviridae (for example, HIV); Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections with these viral agents.

Examples of fungal infections include but are not limited to: aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. The compositions and methods described herein are contemplated for use in treating infections with these fungal agents.

Examples of infectious bacteria include: Helicobacterpyloris, Borelia burgdorferi, Legionella pneumophilia, Mycobacteria sps (such as M. tuberculosis, M. avium, M. intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, and Actinomyces israelli. The compositions and methods described herein are contemplated for use in treating infections with these bacterial agents. Other infectious organisms (such as protists) include: Plasmodium falciparum and Toxoplasma gondii. The compositions and methods described herein are contemplated for use in treating infections with these agents.

In some embodiments of the aspects described herein, the methods further comprise administering an effective amount of a viral, bacterial, fungal, or parasitic antigen in conjunction with an agent for modulating CEACAM1 and TIM3 interactions. Non-limiting examples of suitable viral antigens include: influenza HA, NA, M, NP and NS antigens; HIV p24, pol, gp41 and gp120; Metapneumovirus (hMNV) F and G proteins; Hepatitis C virus (HCV) E1, E2 and core proteins; Dengue virus (DEN1-4) E1, E2 and core proteins; Human Papilloma Virus L1 protein; Epstein Barr Virus gp220/350 and EBNA-3A peptide; Cytomegalovirus (CMV) gB glycoprotein, gH glycoprotein, pp 65, IE1 (exon 4) and pp 150; Varicella Zoster virus (VZV) 1E62 peptide and glycoprotein E epitopes; Herpes Simplex Virus Glycoprotein D epitopes, among many others. The antigenic polypeptides can correspond to polypeptides of naturally occurring animal or human viral isolates, or can be engineered to incorporate one or more amino acid substitutions as compared to a natural (pathogenic or non-pathogenic) isolate.

As used herein, “alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or more as measured by any standard technique. Desirably, the persistent infection is cleared, or pathogen replication has been suppressed, as detected by any standard method known in the art, in which case the persistent infection is considered to have been treated. A patient who is being treated for a persistent infection is one who a medical practitioner has diagnosed as having such a condition. Diagnosis can be by any suitable means. Diagnosis and monitoring can involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired).

As used herein, the terms “treat” “treatment” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as an autoimmune disease, infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of CEA-TIM agent for modulating heterodimeric interactions between a TIM family member and a CEACAM family member needed to alleviate at least one or more symptom of the disease or disorder, and relates to a sufficient amount of pharmacological composition to provide the desired effect, i.e., decrease or increase an immune response, and promote or inhibit T cell tolerance, for example. The term “therapeutically effective amount” therefore refers to an amount of an agent for modulating CEACAM and TIM interactions using the methods as disclosed herein, that is sufficient to effect a particular effect when administered to a typical subject. An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent for modulating interactions between a TIM family member and a CEACAM family member), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The agents useful according to the compositions and methods described herein, including antibodies and other polypeptides, are isolated agents, meaning that the agents are substantially pure and are essentially free of other substances with which they may be found in nature or in vivo systems to an extent practical and appropriate for their intended use. In particular, the agents are sufficiently pure and are sufficiently free from other biological constituents of their hosts cells so as to be useful in, for example, producing pharmaceutical preparations. Because an isolated agent may be admixed with a pharmaceutically acceptable carrier in a pharmaceutical preparation, the agents may comprise only a small percentage by weight of the preparation.

The CEA-TIM specific agents described herein for modulating interactions between a TIM family member and a CEACAM family member can be administered to a subject in need thereof by any appropriate route which results in an effective treatment in the subject. As used herein, the terms “administering,” and “introducing” are used interchangeably and refer to the placement of a polypeptide agent into a subject by a method or route which results in at least partial localization of such agents at a desired site, such as a site of inflammation, such that a desired effect(s) is produced.

In some embodiments, the CEA-TIM specific agents described herein for modulating interactions between a TIM family member and a CEACAM family member are administered to a subject by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the agents for modulating interactions between a TIM family member and a CEACAM family member for use in the methods described herein are administered by intravenous infusion or injection.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a CEA-TIM specific agent for modulating interactions between a TIM family member and a CEACAM family member other than directly into a target site, tissue, or organ, such as a tumor site, such that it enters the subject's circulatory system and, thus, is subject to metabolism and other like processes.

For the clinical use of the methods described herein, administration of a CEA-TIM specific agent for modulating interactions between a TIM family member and a CEACAM family member can include formulation into pharmaceutical compositions or pharmaceutical formulations for parenteral administration, e.g., intravenous; mucosal, e.g., intranasal; ocular, or other mode of administration. In some embodiments, a CEA-TIM specific agent for modulating interactions between a TIM family member and a CEACAM family member described herein can be administered along with any pharmaceutically acceptable carrier compound, material, or composition which results in an effective treatment in the subject. Thus, a pharmaceutical formulation for use in the methods described herein can contain a CEA-TIM specific agent for modulating interactions between a TIM family member and a CEACAM family member as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, aa CEA-TIM specific agent for modulating interactions between a TIM family member and a CEACAM family member. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The CEA-TIM specific agents for modulating interactions between a TIM family member and a CEACAM family member described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (8) nasally. Additionally, a CEA-TIM specific agent can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Further embodiments of the formulations and modes of administration of CEA-TIM specific agents for modulating interactions between a TIM family member and a CEACAM family member that can be used in the methods described herein are illustrated below.

Parenteral Dosage Forms. Parenteral dosage forms of CEA-TIM specific agents for modulating interactions between a TIM family member and a CEACAM family member can also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol formulations. A CEA-TIM specific agent for modulating interactions between a TIM family member and a CEACAM family member can be packaged in a pressurized aerosol container together with suitable propellants, for example, hydrocarbon propellants like propane, butane, or isobutane with conventional adjuvants. A CEA-TIM specific agent for modulating interactions between a TIM family member and a CEACAM family member can also be administered in a non-pressurized form such as in a nebulizer or atomizer. CEA-TIM specific agents for modulating interactions between a TIM family member and a CEACAM family member can also be administered directly to the airways in the form of a dry powder, for example, by use of an inhaler.

Suitable powder compositions include, by way of illustration, powdered preparations of CEA-TIM specific agents for modulating interactions between a TIM family member and a CEACAM family member thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the CEA-TIM specific agents for modulating interactions between a TIM family member and a CEACAM family member described herein further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms. In some embodiments of the methods described herein, CEA-TIM specific agents for modulating interactions between a TIM family member and a CEACAM family member can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound's onset of action, duration of action, plasma levels within the therapeutic window, and peak blood levels. In particular, controlled- or extended-release dosage forms or formulations can be used to ensure that the maximum effectiveness of a compound is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the CEA-TIM specific agents for modulating interactions between a TIM family member and a CEACAM family member described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug.

In some embodiments, CEA-TIM specific agents for modulating interactions between a TIM family member and a CEACAM family member for use in the methods described herein is administered to a subject by sustained release or in pulses. Pulse therapy comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the CEA-TIM specific agents for modulating interactions between a TIM family member and a CEACAM family member administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### EXAMPLES

As demonstrated herein, multiple CEACAM and TIM family members unexpectedly interact and these interactions modulate T cell and iNKT cell functions and activity. More specifically, CEACAM 3, 5, and 6 share >83% or five out of the six interacting residues that were previously shown to be critical for CEACAM1 and TIM-3 interactions, and residues N99, N119, D120, and K122 of TIM3 are conserved in human TIM1 and TIM4. As demonstrated herein, CEACAM1 can form heterodimers not only with human TIM-3, but also with human TIM-1 and human TIM-4 in cis- and/or in trans-configurations.

More specifically, as demonstrated herein, N42 of CEACAM1 and K122 of TIM4, E98 of CEACAM1 and K64 of TIM4, and E99 of CEACAM1 and G62 of TIM4 interact. Further, as demonstrated herein, when human embryonic kidney (HEK) cells are transfected with human CEACAM1 and human TIM-4, their expression tracks together in the same cell, as opposed to when CEACAM1 is co-transfected with integrin α5, wherein such tracking does not occur.

As also demonstrated herein, expression of CEACAM1 facilitates the expression of TIM4 in both primary T cells as well as iNKT cells, and this co-expression is functionally important. CEACAM1 and TIM-4 expression was observed on primary mouse invariant natural killer T cells (iNKT), which are T cells that are restricted to CD1d, a nonclassical MHC class I like molecule. As shown herein, CEACAM1 or TIM-4 deficient (knockout or KO) mice have the same effects on iNKT cells, such that deficiency of either molecule leads to decreased iNKT cell levels. Further, as shown herein, CEACAM1-deficient (Ceacam1−/−) or TIM-4-deficient (Tim4−/−) mice are both protected from an iNKT cell mediated colitis in an identical fashion, indicating that loss of CEACAM1 ligation by TIM-4, and vice versa, is responsible for this phenotype. Moreover, as described herein, CEACAM1 and TIM-4 cross-regulate each other, because upregulation of CEACAM1 and increased T cell deletion were observed in the staphylococcal enterotoxin B induced tolerance model in Tim4−/− mice. These data indicate that TIM-4 normally restrains CEACAM1, presumably, without wishing to be bound or limited by theory, intracellularly, given the studies with TIM-3 and CEACAM1, such that when TIM-4 is deleted it leads to increased CEACAM1 expression and T cell deletion.

The observations described herein help to explain the Janus-like or dual functions of human TIM-1 and TIM-4 described in the literature, as recently shown for human TIM-3 (i.e., TIM-3 is activating in the absence of CEACAM1 and inhibitory in the presence of CEACAM1). TIM-1, for example, has been shown to promote T cell activation and therefore inhibit T cell tolerance induction. Mechanistically, TIM-1 has been suggested to operate as a T cell receptor (TCR) signal substitute which allows T cells to bypass the classical TCR signaling pathway which re-routes it towards co-stimulatory driven activation. However, TIM-1 has also been suggested to be involved in immune inhibition, as TIM-1 functions in promoting the regulatory behavior of B cells, another adaptive arm of immunity that is thought to be important in promoting immune tolerance. This implicates an inhibitory role for TIM-1. This duality of TIM-1 function indicates that there is another ligand for TIM-1 (and TIM-4, a ligand for TIM-1), which determines its inhibitory versus activating functions as observed for TIM-3. Further, studies implicate the importance of the membrane distal N-domain in TIM-1 and TIM-4 function, but it has not been previously demonstrated that any of the TIM family members can interact with each other through this domain. The only known protein ligand to interact with a TIM-related N-domain is CEACAM1, thus supporting the structural predictions described herein that CEACAM1 and other CEACAM family members are common structural partners for the TIM family members, and interact with multiple TIM family members akin to integrins.

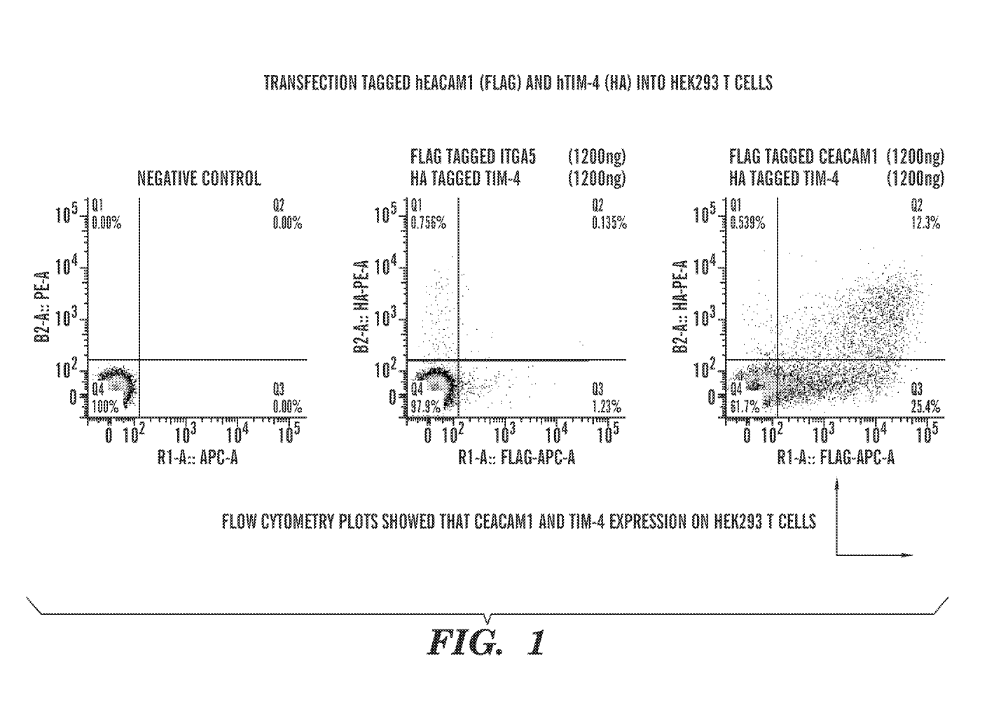

FIG. 1 demonstrates co-expression of human CEACAM1 and human TIM-4 in HEK293T cells, where all hTIM-4 are hCEACAM1-positive.

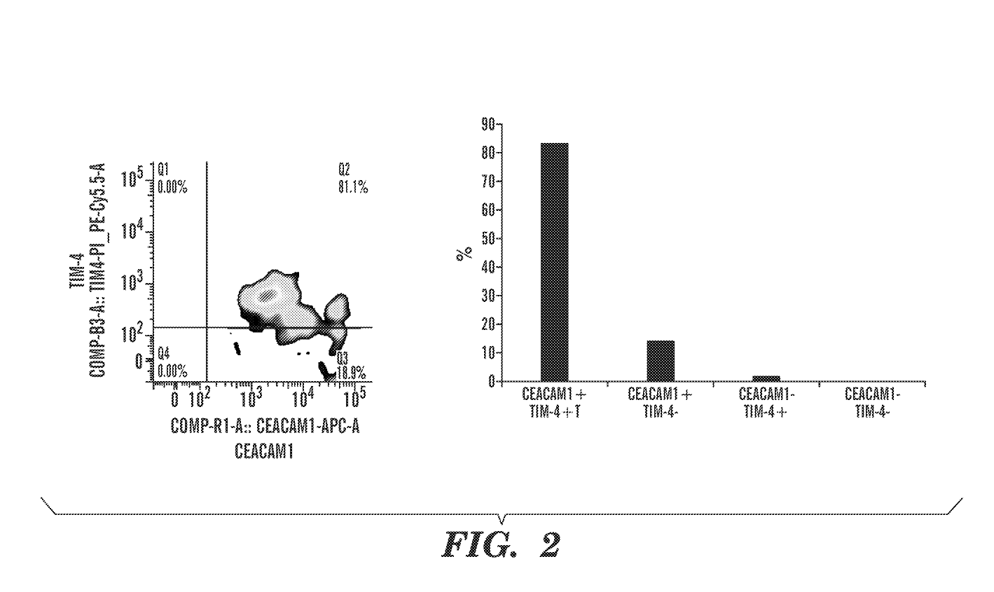

FIG. 2 demonstrates co-expression of CEACAM1 and TIM-4 in colonic infiltrating lamina propria iNKT cells. Representative flow cytometry analyses on the colonic infiltrating iNKT cells the expression of CEACAM1 and TIM-4 under physiological condition. Summary of flow cytometry on the colonic infiltrating iNKT cells and parsed out the cell sub-populations by the expression of CEACAM1 and TIM-3 (n=2, median shown).

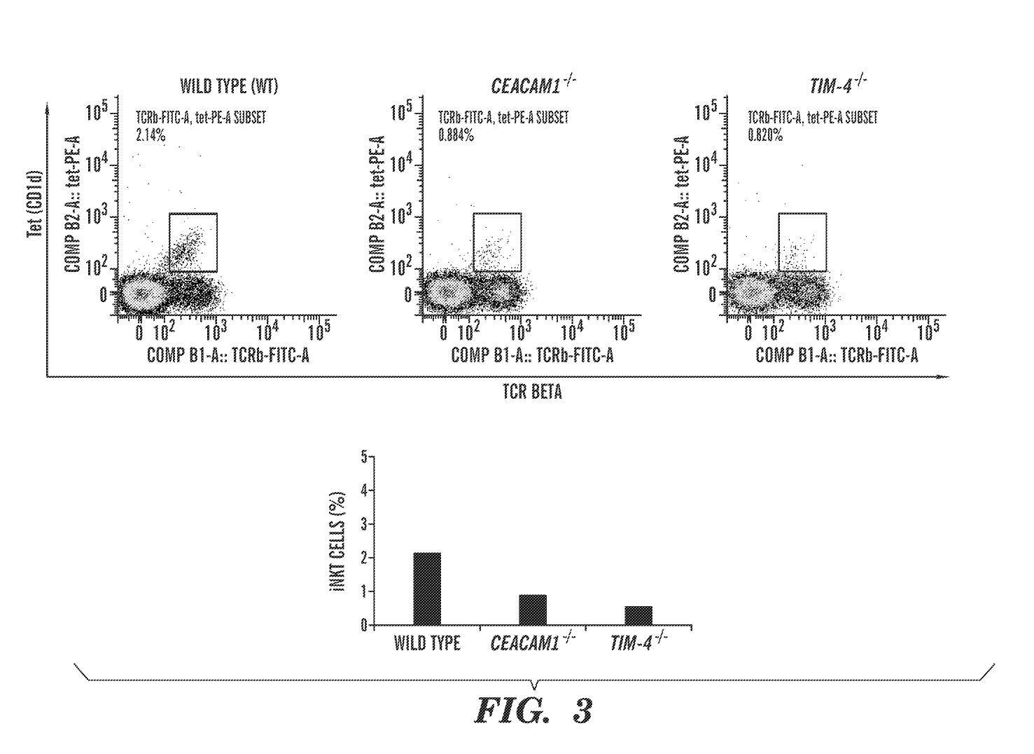

FIG. 3 demonstrates reduced number of iNKT cells in secondary lymphoid organ in CEACAM1- or TIM-4-deficient environment. Representative flow cytometry analyses on the splenic infiltrating iNKT cells under physiological condition shows that the number of iNKT is reduced in Ceacam1−/− and Tim-4−/− mice.

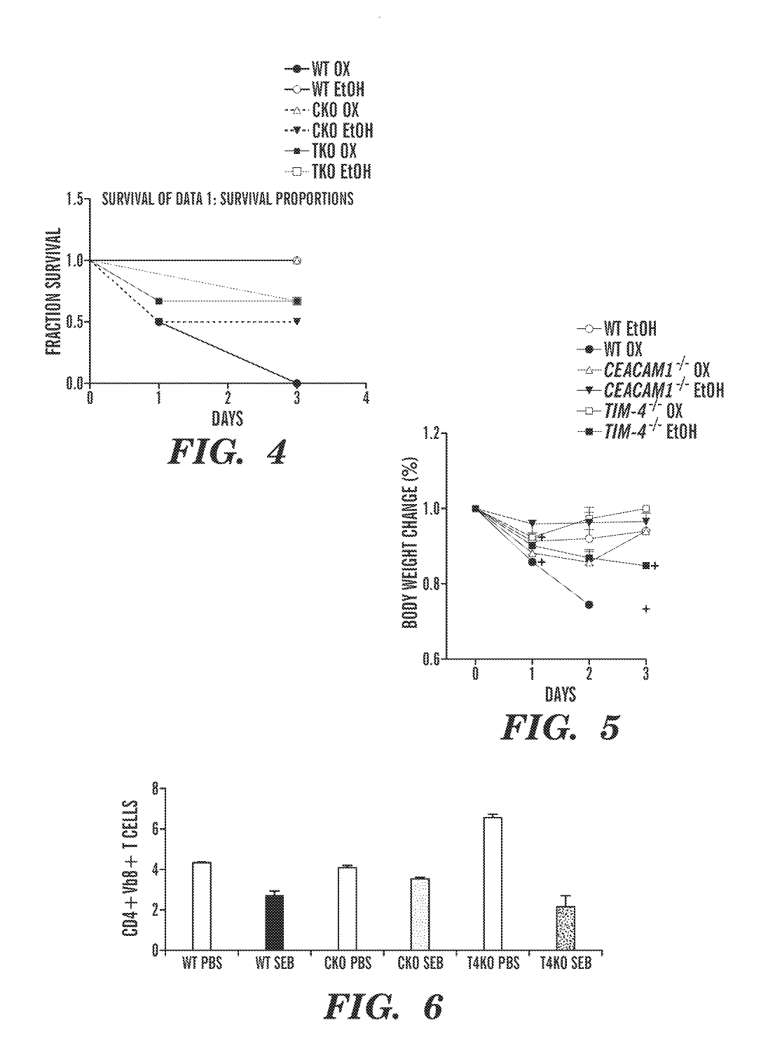

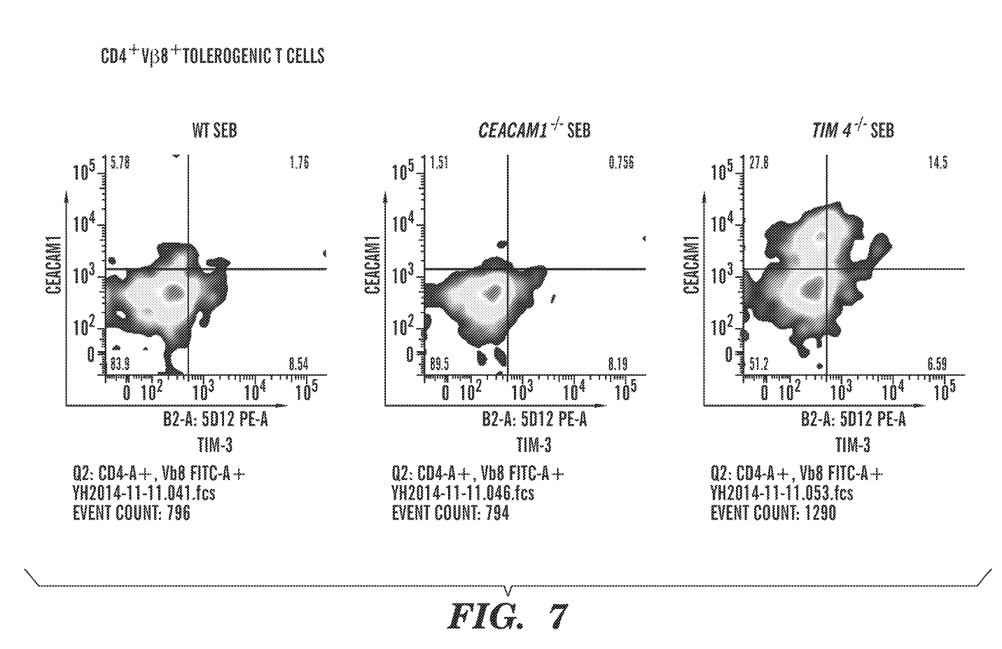

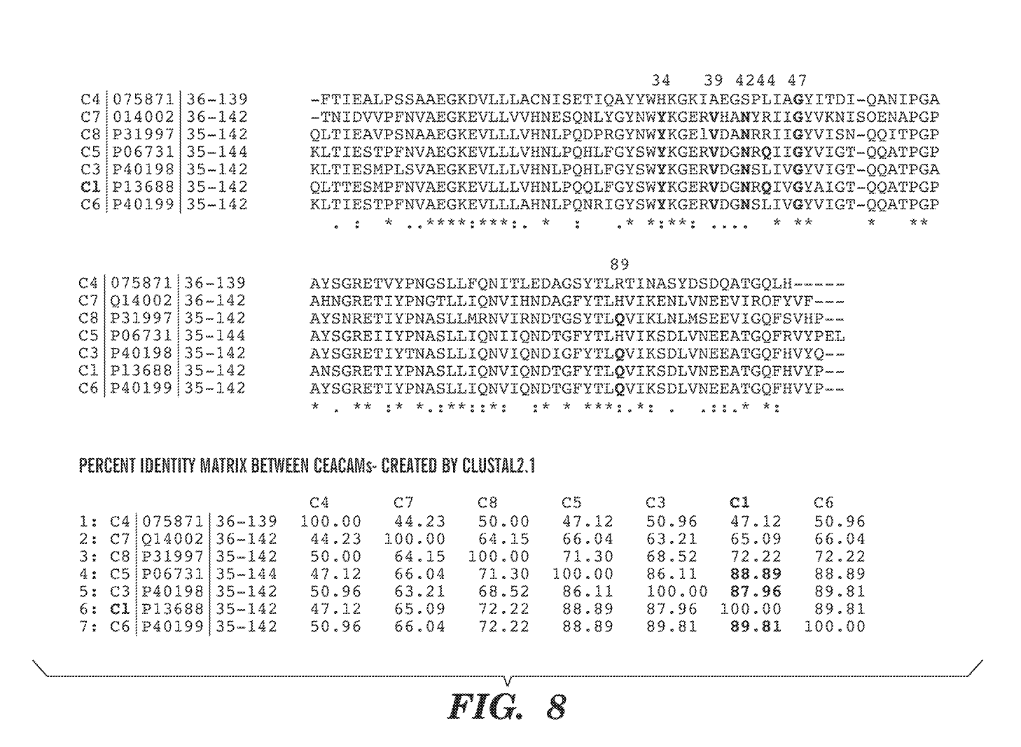

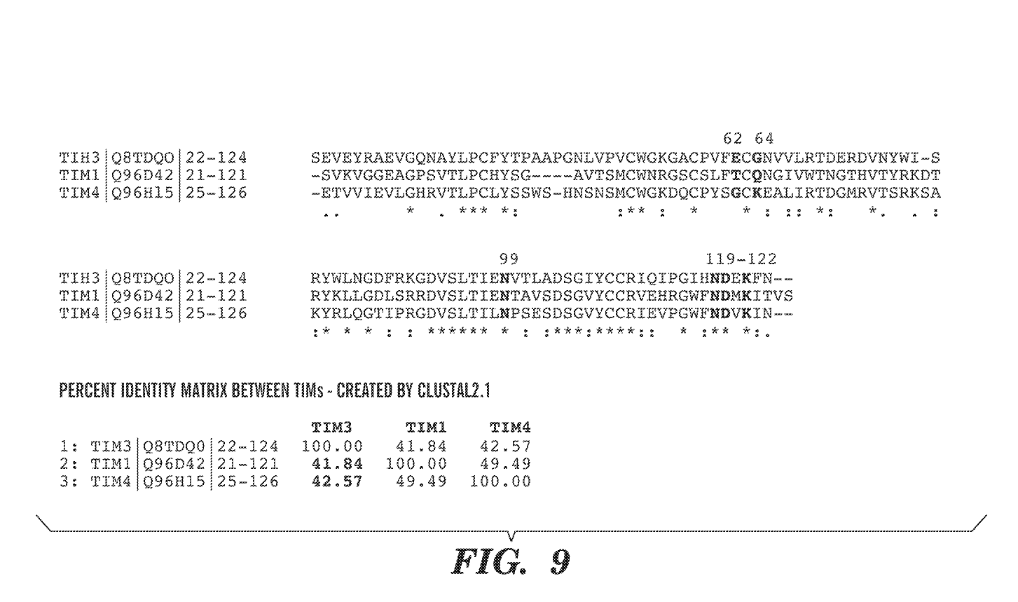

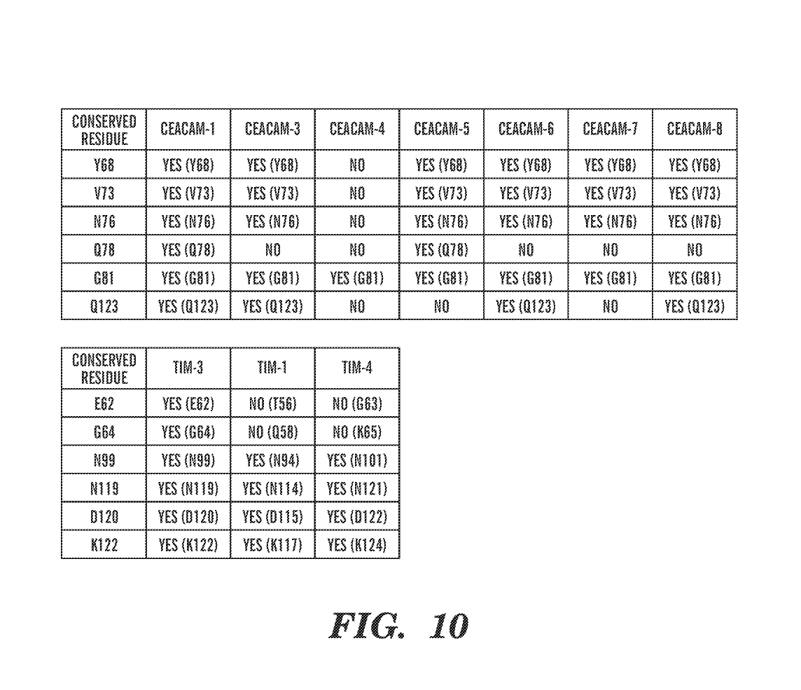

FIG. 4 demonstrates that CEACAM1- and/or TIM-4 can rescue in an iNKT-mediated colitis model. Survival curves in Oxazolone-colitis model are shown. Colorectal challenge was performed with 1% oxazolone (OX) or 50% EtOH (EtOH) as a control (DAY0) 5 days after skin painting with 3% oxazolone. (each group n=2; WT and Ceacam1−/−, n=3; Tim-4−/−)

FIG. 5 demonstrates that CEACAM1- and/or TIM-4 show less severity (body weight loss) in an iNKT-mediated colitis model. Body weight changes in WT, Ceacam1−/− and Tim-4−/− mice in Oxazolone-colitis model are shown.

FIG. 6 demonstrates that TIM-4 deficient mice exhibit excess deletional tolerance in SEB model, and that CEACAM1 is required for deletional tolerance. Bar graph showed the percentage of CD4+Vb8+ T cell in SEB induced tolerance model from WT, Ceacam1−/− (CKO) and Tim4−/− (T4KO) mice which received either PBS control or SEB administration (WT, Ceacam1−/− and Tim4−/− mice) Mouse model of inflammatory bowel disease (IBD)

Splenic mononuclear cells are obtained and CD4+ T cells isolated using anti-CD4 (L3T4) MACS magnetic beads (Miltenyi Biotec, Bergisch Gladbach, Germany) according to the manufacturer's instructions. Enriched CD4+ T cells (94-97% pure as estimated by FACS) are labeled with antibodies. Subpopulations of CD4+ T cells are identified by three-color sorting. All populations are defined as naive and 98.0% pure on re-analysis.

To induce chronic colitis in animals, naive T cells are adoptively-transferred intraperitoneally (i.p.) into 6-10 week-old recipient mice. Weights are measured every week, and mice are sacrificed by CO2 for histological evaluation of colitis and lamina propria mononuclear cells.

### Colorectal Carcinogenesis Models

To examine the effect of CEA-TIM interactions on tumor incidence and multiplicity in an inflammation induced colorectal cancer model, mice are intraperitoneally injected at 6 weeks of age with the carcinogen azoxymethane (AOM) (Sigma-Aldrich) at 10 mg/kg body weight. One week later, the mice are started on the first of two 21-day DSS cycles, consisting of a period of 7 days with the tumor promoter dextran sodium sulfate (DSS) at 2.5% or 1.5% in the drinking water followed by 7 days of receiving regular water. Mice showing signs of morbidity are sacrificed. Colons were removed and flushed with PBS buffer and cut longitudinally. Colon tissue sections are either paraffin-embedded for immunohistochemistry or analysis of lamina propria mononuclear cells associated with the tumors. CT26 colorectal carcinoma cells are diluted into 10×106 cell/ml of PBS. After shaving, mice receive 200 μl of the CT26 cell suspension subcutaneously in the left flank. All treatments with CEA-TIM specific agents are applied intraperitoneally into the right flank

Tumor growth is assessed three times per week at seven days after tumor inoculation by measuring the width and length of the tumors (mm) using calipers and the areas defined (mm2; width×length). Mice are sacrificed based upon the extent of tumor growth observed in the control agent treated control animals to minimize animal suffering in accordance with the approved animal protocol. Histopathological examination of colitis and colitis-associated colorectal cancer

Colons are removed from mice after termination and dissected free from the anus to the cecum. Colonic contents are removed and colons cleaned with PBS prior to fixation in 4% paraformaldehyde or 10% neutral buffered formalin followed by routine paraffin embedding. After paraffin embedding, 0.5 mm sections were cut and stained with hematoxylin and eosin. Sections are examined and colitis scored in a blinded fashion (with respect to genotype and experimental protocol). Each of four histologic parameters for the severity of colitis is scored as absent (0), mild to severe (1-6): mononuclear cell infiltration, polymorphonuclear cell infiltration, epithelial hyperplasia, epithelial injury and extent of inflammation. For AOM-DSS, colitis-associated colorectal cancer studies, all tissues are examined for dysplasia, adenoma, low-to-high-grade adenoma and adenocarcinoma in a blinded fashion.

The expression of CEACAM family members and TIM family members in the spleens, peripheral blood cells, and lymphoid tissues of these mice is also examined. Tumor infiltrating lymphocytes are isolated by dissociating tumor tissue in the presence of collagenase D (25 mg/ml) for 20 min prior to centrifugation on a discontinuous PERCOLL gradient (GE Healthcare). Isolated cells are then used in various assays of T cell function. In particular, the frequency of CD8⁺ cells that co-express CEACAM family members and TIM family members is examined. Additionally, the expression of CEACAM family members and TIM family members is examined in CD8⁺ TIL subpopulations, such as CD44 and CD62L cells. Expression of CEACAM family members and TIM family members indicates the functional states of these cells. Further, the expression (i.e., secretion) of cytokines, such as IL-2, TNFα and IFNγ can be measured ex vivo, for example by flow cytometry. Single cell suspensions are stained with antibodies against, for example, CD4, CD8, CD44, CD62L, CD66, and desired CEACAM family members and TIM family members. 7AAD is used to exclude dead cells. For intracytoplasmic cytokine staining, cells are stimulated in vitro with PMA (50 ng/ml) and ionomycin (1 μg/ml) for 3 hr in the presence of Golgi plug (BD BIOSCIENCES). Cells are then harvested and stained with CD8, and antibodies to the desired CEACAM family members and TIM family members prior to fixation and permeabilization. Permeabilized cells are then stained for IL-2 (JES6-5H4), TNFα (MP6-XT22) and IFNγ (XMG1.2). All data are collected on a BD LSRII (BD Biosciences) and analyzed with FLOWJO Software (TREE STAR). For example, CEACAM-1⁺TIM-3⁺ cells are typically associated with TIL exhaustion, indicated by impaired IFNγ production.

Additionally, the loss of the ability to proliferate in response to TCR stimulation is among the effector functions lost in tolerant T cells. The ability of TILs to proliferate directly ex vivo is examined by determining expression of Ki-67, a nuclear protein expressed by cells that have entered into the cell cycle. It has been noted, however, that in individuals chronically infected with HIV, cells that are arrested in G1 can express Ki-67 (Combadere et al., 2000). DNA content is also examined by simultaneously staining with TO-PRO-3 iodide. By doing so, cells arrested in G1 can be discerned from cells that have progressed to S, G2 and M phase. TILs are isolated and stimulated directly ex vivo prior to examination of Ki-67 expression and DNA content. TILs are harvested and cultured in vitro in the presence of anti-CD3 (1 μg/ml) for 48 hrs. Cells are then stained with antibodies against CD8, and the desired CEACAM family members and TIM family members prior to permeabilization and staining with antibody against Ki-67 (BIOLEGEND) and with TO-PRO-3 iodide (INVITROGEN). All data are collected on a BD LSRII (BD BIOSCIENCES) and analyzed with FLOWJO software (TREE STAR). The abundance of cells co-expressing various combinations of CEACAM family members and TIM family members in G0, G1 and S-M phases of cell cycle is then determined.

To test whether combined targeting of CEACAM family members and TIM family members can restore anti-tumor immunity in vivo, the expression of the desired CEACAM family members and TIM family members on lymphocytes in CT26 tumor-bearing mice is confirmed. CT26 tumor-bearing mice are then treated with antibodies to the desired CEACAM family members and TIM family members, alone and in combination, or with control immunoglobulins. For example, mice are treated with either 100 μg of anti-TIM-4 antibody i.p. on days 0, 2 and 4; or 100 μg of anti-CEACAM-1 antibody i.p. on days 0, 3, 6, 9 and 12, or a mixture of anti-TIM-4 and anti-CEACAM-1 antibodies on the same days, or isotype control immunoglobulins (Rat IgG1 and RatIgG2b). These treatments are then compared. Reduction in tumor growth, reflected in both tumor size and efficacy among the test subjects is indicative of restoration of anti-tumor immunity.

Similarly, the effects of combined targeting of CEACAM family members and TIM family members can be tested in mice bearing B16 melanoma relative to control immunoglobulin-treated mice.

To address directly whether combined targeting of CEACAM family members and TIM family members indeed restores TILs function, TILs from mice bearing CT26 tumor are isolated and cultured in the presence of antibodies to various combinations of CEACAM family members and TIM family members, or control immunoglobulins. TILs are harvested and cultured (1-3×10⁵/well) in the presence of soluble anti-CD3 (5 μg/ml) and antibodies to CEACAM family members and TIM family members, alone or in combination, or control immunoglobulins. After 96 hr, culture supernatants are collected and IFNγ measured by cytometric bead array (CBA) (BD BIOSCIENCES). Additionally, the effect of combined targeting of CEACAM family members and TIM family members on peripheral T-cell responses from tumor-bearing mice can also be examined. More specifically, for instance, cells from the tumor draining lymph node of treated mice are cultured with the tumor antigen AH1 (30 μg/ml). Supernatant is collected at 48 hr, and production of IFNγ assessed.

### Experimental Autoimmune Encephalomyelitis

SJL mice are immunized with 100 μg of PLP 139-151 emulsified in either complete Freund's adjuvant (CFA) supplemented with Mycobacterium tuberculosis (4 mg/ml), incomplete Freund's adjuvant (IFA) containing antibodies to various CEACAM family members and TIM family members, alone and in combination, isotype controls, or IFA alone. Mice are also administered 100 ng pertussis toxin (List) intravenously on days 0 and 2. All antibodies used in vivo are LPS free. Mice are monitored daily for the development of disease, which is scored according to the following scale: 0, no clinical signs; 1, loss of tail tone; 2, hindlimb weakness; 3, hindlimb paralysis; 4, forelimb paralysis; and 5, moribund or dead. EAE symptoms are generally exacerbated by agents that inhibit T cell tolerance. For example, in view of the discovery disclosed herein that CEACAM-1 is a ligand for TIM-4, it is considered that treatment with anti-TIM-4 inhibitory antibody and anti-CEACAM-1 inhibitory antibody, either by co-administration or in the form of a bispecific polypeptide agent, will synergistically improve EAE symptoms or disease markers. In contrast, co-administration of agents that activate TIM-4 and CEACAM-1-mediated signaling or of a bispecific agent that activates TIM-4/CEACAM-1 mediated signaling is contemplated to synergize for exacerbation of EAE symptoms or disease markers.

1. A composition for modulating a heterodimeric interaction between TIM-3 and CEACAM-1 comprising a CEA-TIM specific agent that specifically binds to TIM-3, CEACAM-1, or both TIM-3 and CEACAM-1 and inhibits signaling mediated by the interaction of TIM-3 and CEACAM-1.

2. A composition for modulating a heterodimeric interaction between TIM-3 and CEACAM-1 comprising a CEA-TIM specific agent that specifically binds to TIM-3, CEACAM-1, or both TIM-3 and CEACAM-1 and increases signaling mediated by the interaction of TIM-3 and CEACAM-1.

3. The composition of any one of claims 1-2, wherein the CEA-TIM specific agent binds TIM-3 at one or more amino acid residues selected from C52, C63, G64, N99, T101, C109, N119, and K122 of SEQ ID NO: 3

4. The composition of any one of claims 1-3, wherein the CEA-TIM specific agent binds CEACAM-1 at one or more amino acid residues selected from Y68, V73, N76, R77, Q78, G81, Q123, E132, and E133 of SEQ ID NO: 4.

5. The composition of any one of claims 1-4, wherein the CEA-TIM specific agent comprises an antibody or antigen binding portion thereof, a peptide or fusion protein, or a small molecule compound.

6. The composition of any one of claims 1-4, wherein the CEA-TIM specific agent comprises a soluble TIM family member or a functional fragment or peptide variant thereof.

7. The composition of any one of claims 1-4, wherein the CEA-TIM specific agent comprises a soluble CEACAM family member or a functional fragment or peptide variant thereof.

8. A method for promoting T-cell tolerance in a subject in need thereof, the method comprising administering a therapeutically effective amount of a CEA-TIM specific agent that binds TIM-3 and/or CEACAM-1 and increases signaling mediated by the interaction of TIM-3 and CEACAM-1 of any one of claims 2-7.

9. The method of claim 8, wherein the subject has an autoimmune disease

10. The method of claim 8, wherein the subject is an organ or transplant recipient.

11. A method for decreasing T-cell tolerance or decreasing T-cell functional exhaustion or increasing an immune response in a subject in need thereof, the method comprising administering a therapeutically effective amount of a CEA-TIM specific agent that binds TIM-3 and/or CEACAM-1 and decreases signaling mediated by the interaction of TIM-3 and CEACAM-1 of any one of claims 1 and 3-7.

12. The method of claim 11, wherein the subject has a cancer or a tumor.

13. The method of claim 11, wherein the subject has a chronic infection.

14. Use of a CEA-TIM specific agent that binds TIM-3 and/or CEACAM-1 and increases signaling mediated by the interaction of TIM-3 and CEACAM-1 of any one of claims 2-7 for promoting T-cell tolerance in a subject in need thereof.

15. The use of claim 14, wherein the subject has an autoimmune disease

16. The use of claim 14, wherein the subject is an organ or transplant recipient.

17. Use of a CEA-TIM specific agent that binds TIM-3 and/or CEACAM-1 and decreases signaling mediated by the interaction of TIM-3 and CEACAM-1 of any one of claims 1 and 3-7 for decreasing T-cell tolerance or decreasing T-cell functional exhaustion or increasing an immune response in a subject in need thereof.

18. The use of claim 17, wherein the subject has a cancer or a tumor.

19. The use of claim 17, wherein the subject has a chronic infection.
